(19) United States
(12) Patent Application Publication
(10) Pub. No.: US 2024/0139779 A1
(43) Pub. Date: May 2, 2024

(54) SYSTEMS AND METHODS FOR VISUAL VERIFICATION SYSTEM OF MEDICATION, MEDICATION PRESCRIPTION VERIFICATION, AND ALTERNATIVE MEDICATION RECOMMENDATION

(71) Applicant: Mediosis, LLC, Coppell, TX (US)

(72) Inventors: Venkat Rao, Southlake, TX (US); Jacob Awkal, Plano, TX (US)

(73) Assignee: Mediosis, LLC, Coppell, TX (US)

(21) Appl. No.: 18/500,080

(22) Filed: Nov. 1, 2023

Related U.S. Application Data
(60) Provisional application No. 63/382,016, filed on Nov. 2, 2022.
(60) Provisional application No. 63/382,054, filed on Nov. 2, 2022.
(60) Provisional application No. 63/382,063, filed on Nov. 2, 2022.
(60) Provisional application No. 63/382,064, filed on Nov. 2, 2022.

(51) Int. Cl. B07C 5/342; B07C 5/02; G06V 10/12; G06V 10/44; G06V 10/774; G06V 10/82; G06V 20/52

(52) U.S. Cl. CPC B07C 5/3425; B07C 5/02; G06V 10/12; G06V 10/44; G06V 10/774; G06V 10/82; G06V 20/52

(57) ABSTRACT

Disclosed are a visual verification system for medication, medication prescription verification, and alternative medication recommendation. A visual verification system may include a medication sorting device that has a medication entry; a moving part configured to receive the plurality of medications from the medication entry at a first location and move the plurality of medications to a second location; a fixed part disposed adjacent the moving part and configured to interact with the plurality of medications, as the plurality of medications are moved to the second location, in order to generate a sequence of axially spaced apart medications. For instance, a method may include receive, from camera(s), a plurality of images of a plurality of medications; determine, based on the plurality of images, features of at least a first medication of the plurality of medications; and determine an identity of at least the first medication based on the features.

### CROSS-REFERENCE TO RELATED APPLICATION(S)

This application claims the benefit of priority under 35 U.S.C. §§ 120 and 119(e) to (1) U.S. Provisional Application No. 63/382,016, filed Nov. 2, 2022; (2) U.S. Provisional Application No. 63/382,054, filed Nov. 2, 2022; (3) U.S. Provisional Application No. 63/382,063, filed Nov. 2, 2022; and (4) U.S. Provisional Application No. 63/382,064, filed Nov. 2, 2022. The entire contents of each of which are incorporated herein by reference.

### TECHNICAL FIELD

Various aspects of the present disclosure relate generally to a visual verification system for medication, medication prescription verification, and alternative medication recommendation and, more generally, to systems and methods for an end-to-end communication architecture to manage medication fulfillment.

### BACKGROUND

Generally, prescribing and fulfilling medications is a complicated process, as each patient, healthcare provider, pharmacist, and network has different circumstances. For instance, patients may have data about their medications, conditions, and health status in various systems; healthcare providers may have subsets of such data but not all such data, and may not be aware of medication availability, recalls, or interactions; pharmacists may have subsets of such data but not all such data, and may not be aware of patient history or medications. As such, medication prescription process flows and alternative recommendations may be a challenge.

Moreover, prescriptions/over-the-counter medication fulfillment may be a complicated process, due to concerns with infectious disease (e.g., SARS-CoV-2, or other diseases) or due to exposures between medications in medication verification systems. For instance, historically, pharmacists individually verified medications in a prescription. However, recent trends have required pharmacists to review images of samples of medication before providing the same to patients. Furthermore, to the extent current systems incorporated automated systems, the automated systems were not based on individual medication (pill or capsule) identification or record keeping. Therefore, patients were subject to healthcare risks if individual medications could not be individually verified by trained users. As such, efficient and accurate visual verification systems are a challenge.

The present disclosure is directed to overcoming one or more of these above-referenced challenges.

### SUMMARY OF THE DISCLOSURE

According to certain aspects of the disclosure, systems, methods, and computer readable memory are disclosed for a visual verification system for medication, medication prescription verification, and alternative medication recommendation.

For instance, a medication sorting device may include: a medication entry configured to receive a plurality of medications; a moving part configured to receive the plurality of medications from the medication entry at a first location and move the plurality of medications to a second location; a motor configured to move the moving part; and a fixed part disposed adjacent the moving part and configured to interact with the plurality of medications, as the plurality of medications are moved to the second location, in order to generate a sequence of axially spaced apart medications.

Furthermore, a system for medication visual verification may include: a channel configured to receive a plurality of medications; one or more cameras, the one or more cameras being configured to generate a plurality of images of the plurality of medications as the medications travel through the channel; at least one processor; and at least one memory storing instructions. The system may be configured to: receive, from the one or more cameras, the plurality of images of the plurality of medications; determine, based on the plurality of images, features of at least a first medication of the plurality of medications; and determine an identity of at least the first medication based on the features.

Moreover, a system for prescription data verification may include: at least one processor; and at least one memory storing instructions. The system may be configured to: receive, from a user device associated with a medical provider, prescription data for a proposed prescription for a patient; extract a medication identifier, usage indication, prescription directions, a quantity amount, a medication strength, and patient data for the patient from the prescription data; determine a user identifier that corresponds to the patient based on the patient data; retrieve active prescriptions and existing health conditions for the patient; perform an analysis to flag for verification by the medical provider, including one or combinations of checks to: determine whether a medication corresponding to the medication identifier is under a recall flag or associated with the usage indication; determine whether the medication has contraindications with the active prescriptions or interactions with the existing health conditions; determine whether the prescription directions, the quantity amount, and the medication strength satisfy conditions associated with the medication; determine whether the medication is a pharmacogenomically labelled medication; and/or determine whether the medication, in the quantity amount and the medication strength, is available at a set of pharmacies; in response to any check of the checks returning an issue, transmit a notification to the user device associated with the medical provider to receive a verification of the prescription data or a change in the prescription data; and in response to no checks returning an issue, transmit a prescription request to a prescription management system, so that a prescription for the patient may be fulfilled.

Furthermore, a system for recommending alternative medications may include: at least one processor; and at least one memory storing instructions. The system may be configured to: receive, from a user device associated with a medical provider, prescription data for a proposed prescription for a patient; extract a medication identifier, usage indication, prescription directions, a quantity amount, a medication strength, and patient data for the patient from the prescription data; determine a user identifier that corresponds to the patient based on the patient data; retrieve active prescriptions and existing health conditions for the patient; perform an analysis to flag for verification by the medical provider, including one or combinations of checks to: determine whether a medication corresponding to the medication identifier is under a recall flag or associated with the usage indication; determine whether the medication has contraindications with the active prescriptions or interactions with the existing health conditions; determine whether the prescription directions, the quantity amount, and the medication strength satisfy conditions associated with the medication; determine whether the medication is a pharmacogenomically labelled medication; and/or determine whether the medication, in the quantity amount and the medication strength, is available at a set of pharmacies; in response to any check of the checks returning an issue, transmit a notification to the user device associated with the medical provider to recommend a different medication, a different prescription direction, a different quantity amount, a different medication strength, or a genotyping test; and in response to no checks returning an issue, transmit a prescription request to a prescription management system, so that a prescription for the patient may be fulfilled.

Additional objects and advantages of the disclosed technology will be set forth in part in the description that follows, and in part will be apparent from the description, or may be learned by practice of the disclosed technology.

It is to be understood that both the foregoing general description and the following detailed description are exemplary and explanatory only and are not restrictive of the disclosed technology, as claimed.

### BRIEF DESCRIPTION OF THE DRAWINGS

The accompanying drawings, which are incorporated in and constitute a part of this specification, illustrate various exemplary aspects and together with the description, serve to explain the principles of the disclosed technology.

FIG. 1 shows an example environment 100 for a visual verification system for medication, medication prescription verification, and alternative medication recommendation.

FIGS. 2A-2D depict external features of the example visual verification system 125.

FIGS. 3A-3D depict a sorting features of the example visual verification system 125.

FIGS. 4A-4E depict visual verification features of the example visual verification system 125.

FIG. 5 depicts an operation of the example visual verification system 125.

FIGS. 6A-6B depict a user interface of the example visual verification system 125.

FIG. 7 depicts operations for medication prescription verification through to medication fulfillment.

FIG. 8 depicts operations for alternative medication recommendation through to medication fulfillment.

FIG. 9 depicts an example system that may execute techniques presented herein.

### DETAILED DESCRIPTION

In general, the present disclosure is directed to methods and systems for a visual verification system for medication, medication prescription verification, and alternative medication recommendation. As discussed in detail herein, visual verification systems of the present disclosure may automatically image, ID, and sort medications without human interaction, thereby reducing exposure risk while increasing accuracy. Moreover, certain methods may verify data (names, addresses, and the like, thereby reducing insurance delays) and check both medical conflicts and availability conflicts, thereby increasing quality of care for patients, reducing time spent by users (e.g., re-entering or adjusting prescriptions after issues are discovered), and reducing delay to when a prescription can be fulfilled for a patient.

Thus, methods and systems of the present disclosure may be improvements to computer technology and/or a visual verification system for medication, medication prescription verification, and alternative medication recommendation.

Environment

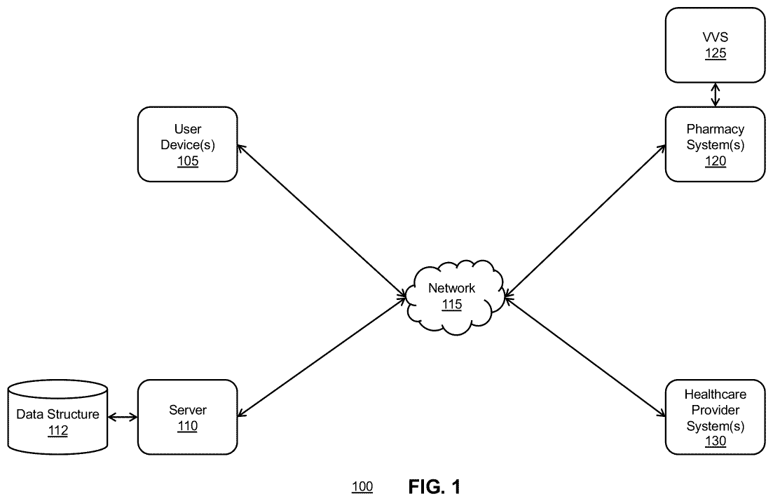

FIG. 1 shows an example environment 100 for a visual verification system for medication, medication prescription verification, and alternative medication recommendation. The environment 100 may include user device(s) 105, a server 110, data structure 112, network 115, pharmacy system(s) 120, visual verification system 125, and healthcare provider system(s) 130.

The user device(s) 105 (hereinafter “user device 105” for ease of reference) may be a personal computing device, such as a cell phone, a tablet, a laptop, or a desktop computer. In some cases, the user device 105 may be an extended reality (XR) device, such as a virtual reality device, an argument reality device, a mixed reality device, and the like. In some cases, the user device 105 may be associated with a user (e.g., a patient) of medical services. The user may have a user account associated with the server 110 that uniquely identifies a user. Generally, the user device 105 may execute an application (e.g., a browser program, a mobile application, a desktop application, and the like) that enables the user device 105 to interact with the server 110, the pharmacy system(s) 120, and the healthcare provider system(s) 130, as discussed herein.

The network 115 may include one or more local networks, private networks, enterprise networks, public networks (such as the internet), cellular networks, and satellite networks, to connect the various devices in the environment 100. Generally, the various devices of the environment 100 may communicate over network 115 using, e.g., network communication standards that connect endpoints corresponding to the various devices of the environment 100.

The pharmacy system(s) 120 (hereinafter “pharmacy system 120” for ease of reference) may be a personal computing device, such as a cell phone, a tablet, a laptop, or a desktop computer. In some cases, the pharmacy system 120 may be an extended reality (XR) device, such as a virtual reality device, an argument reality device, a mixed reality device, and the like. In some cases, the pharmacy system 120 may be associated with a specific pharmacy (e.g., a independent pharmacy) or a network of such pharmacies (e.g., co-managed at multiple locations), each of which may store and provide medications to patients based on prescriptions. The pharmacy or network of pharmacies may each have one or a plurality of users (e.g., pharmacists, technicians, and the like) that may interact with a shared or individual pharmacy account associated with the server 110 that uniquely identifies the pharmacy or network of pharmacies. In some cases, the users may have separate or shared credentials to interact with the server 110. Generally, the pharmacy system 120 may execute an application (e.g., a browser program, a mobile application, a desktop application, and the like) that enables the users of the pharmacy system 120 to interact with the server 110, the user device(s) 105, and the healthcare provider system(s) 130, as discussed herein.

The healthcare provider system(s) 130 (hereinafter “healthcare provider system 130” for ease of reference) may be a personal computing device, such as a cell phone, a tablet, a laptop, or a desktop computer. In some cases, the healthcare provider system 130 may be an extended reality (XR) device, such as a virtual reality device, an argument reality device, a mixed reality device, and the like. In some cases, the healthcare provider system 130 may be associated with a specific healthcare enterprise (e.g., a healthcare provider) or a network of such healthcare enterprises (e.g., co-managed at multiple locations), each of which may provide healthcare services to patients, including prescribing medications. The healthcare enterprise or network of healthcare enterprises may each have one or a plurality of users (e.g., doctors, technicians, nurses, and the like) that may interact with a shared or individual healthcare account associated with the server 110 that uniquely identifies the healthcare enterprise or network of healthcare enterprises. In some cases, the users may have separate or shared credentials to interact with the server 110. Generally, the healthcare provider system 130 may execute an application (e.g., a browser program, a mobile application, a desktop application, and the like) that enables the users of the healthcare provider system 130 to interact with the server 110, the user device(s) 105, and the pharmacy system(s) 120, as discussed herein.

The visual verification system 125 (“WS 125”) may be operated by one or more users (or users associated with those users) of the pharmacy system 120. In some cases, there may be one or a plurality of visual verification system 125 at a location of the pharmacy system 120. As discussed herein, a visual verification system 125 may assist users of the pharmacy system 120 with medication fulfillment, inventory management, and/or safety/quality control (e.g., ensuring what is in a given storage container or prescription is what it purports to be).

The server 110 may manage data and communications between the user device(s) 105, the healthcare provider system(s) 130, and the pharmacy system(s) 120, as discussed herein. In certain aspects, the server 110 may execute a server-side application (e.g., corresponding to a browser program, a mobile application, a desktop application, and the like on the pharmacy system 120, the user device(s) 105, and the healthcare provider system(s) 130) so that instructions, requests, and messages may be stored, managed, and transmitted to relevant endpoints, as discussed herein. The server 110 may store relevant data (as discussed herein) in the data structure 112. The data structure 112 may be a structured or unstructured database or other data storage system (e.g., time series database). As one would recognize, the data structure 112 may store both data (in various tables or data structures, in different secure methodologies based on sensitivity, etc.) and rules (as discussed herein, as updated over time). In some cases, the server 110 may rely communications between components of environment while also providing feedback and/or records for reference by each of the various components. More details of the feedback and/or record functions of the server 110 are discussed below with respect to FIG. 1, 2A-2D, 3A-3D, 4A-4E, 5, 6A-6b, 7, and 8.

Visual Verification System

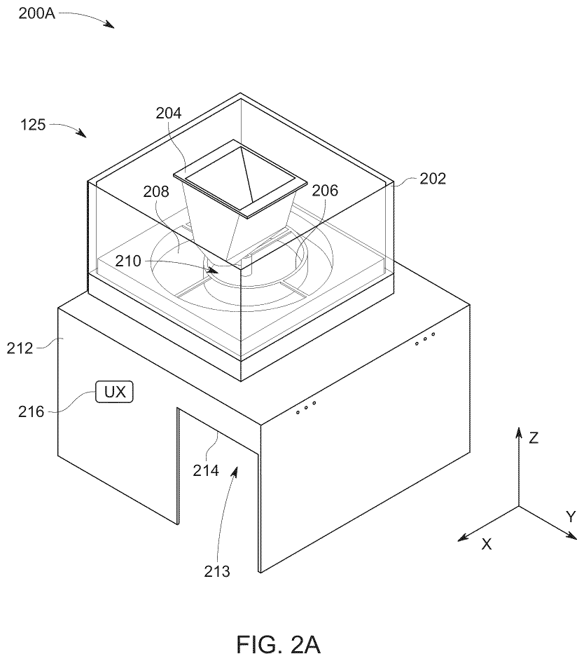

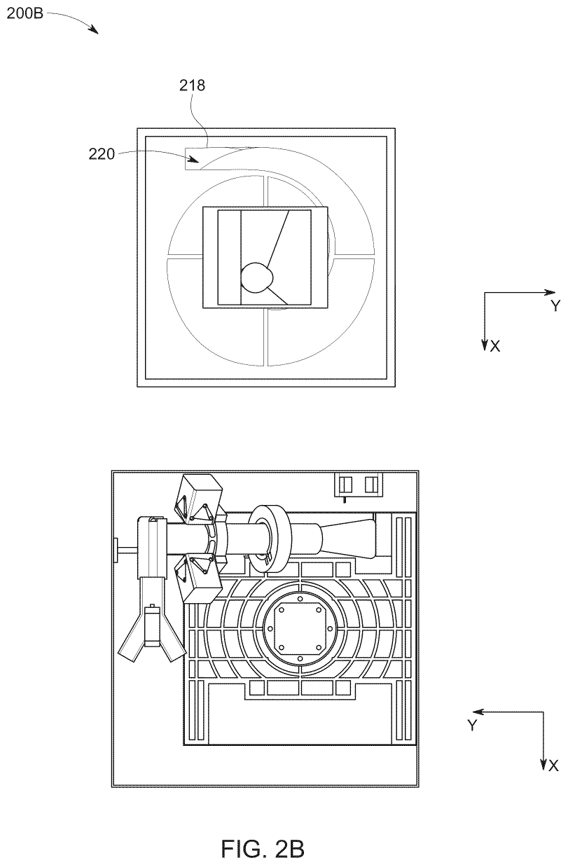

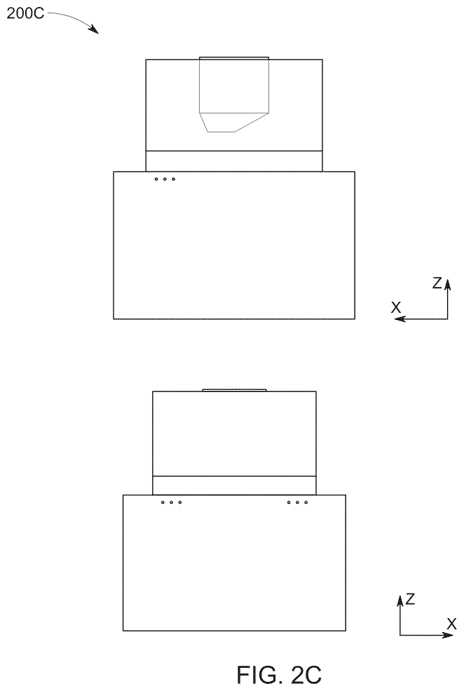

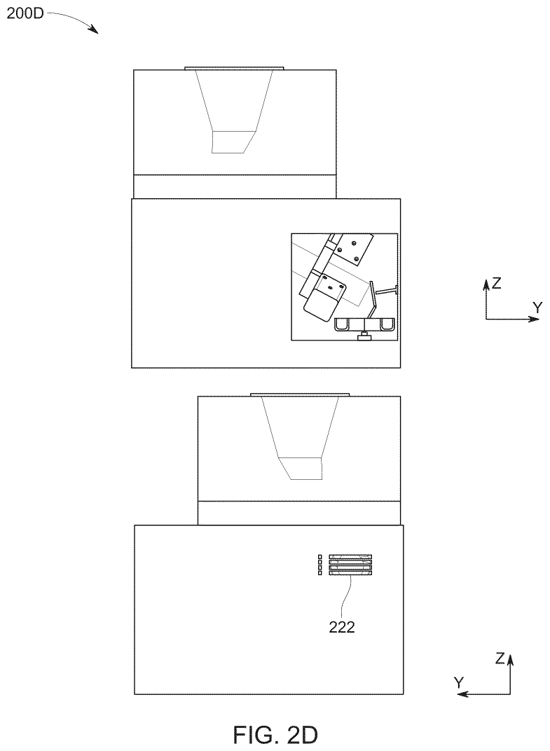

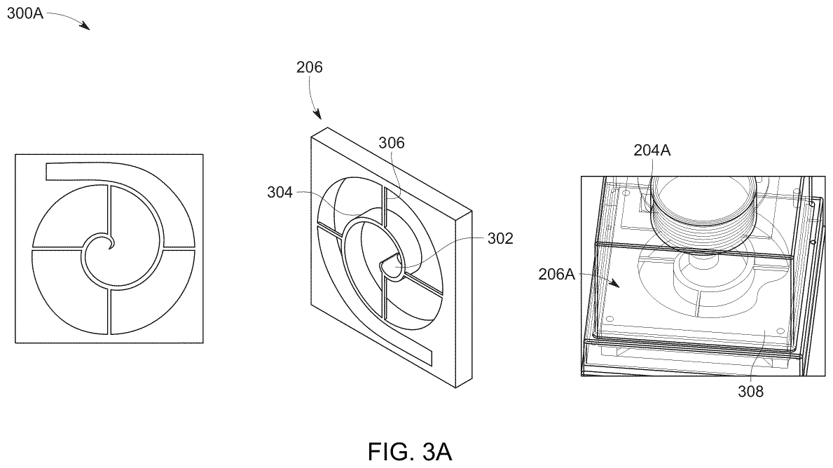

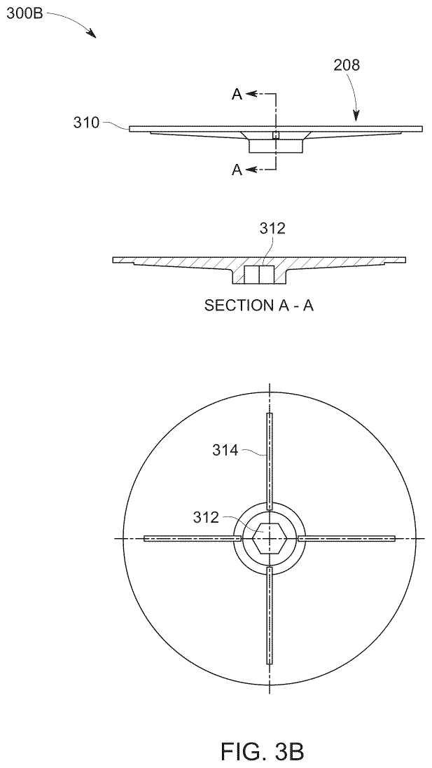

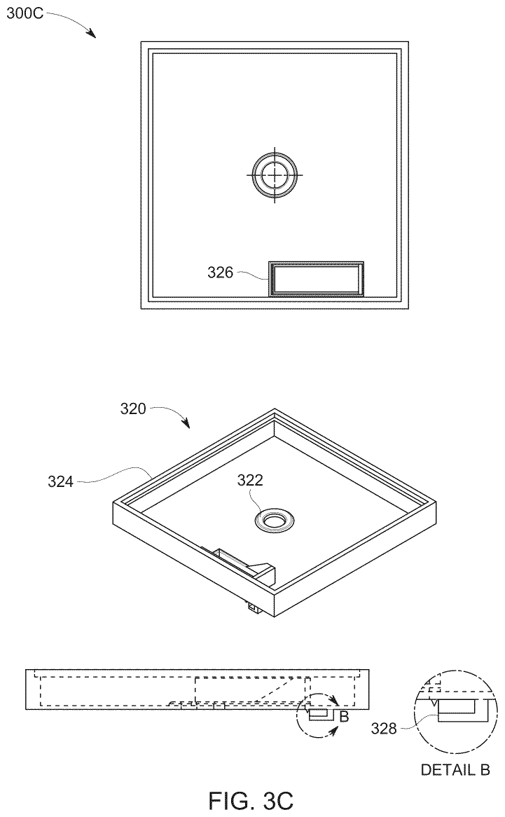

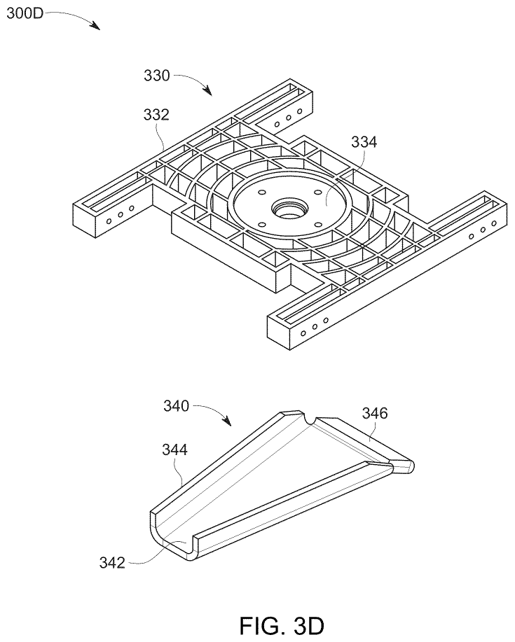

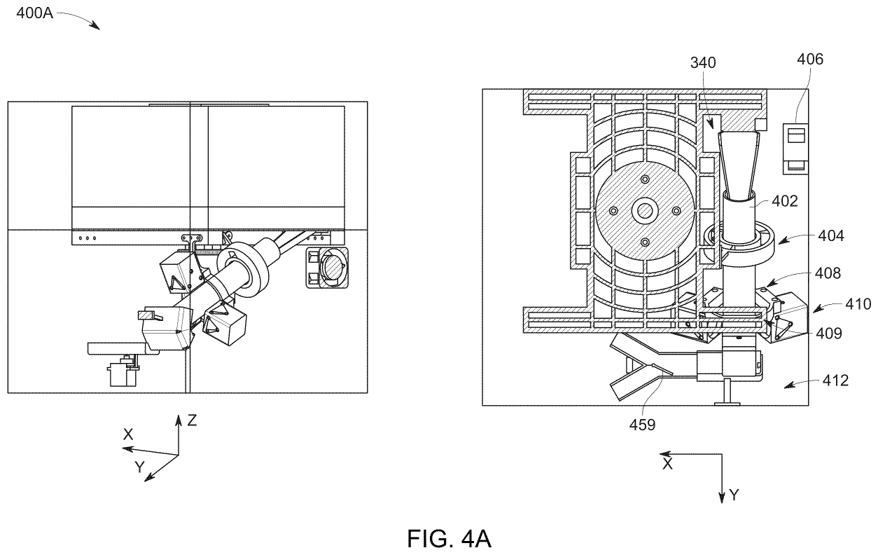

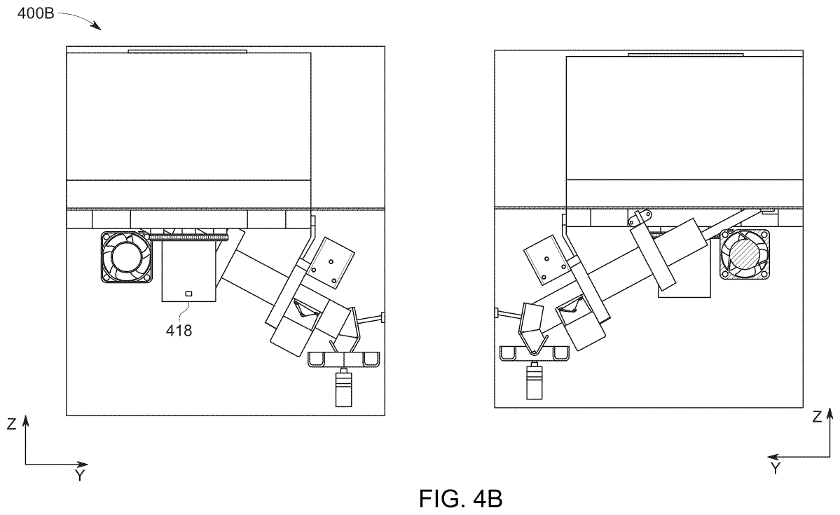

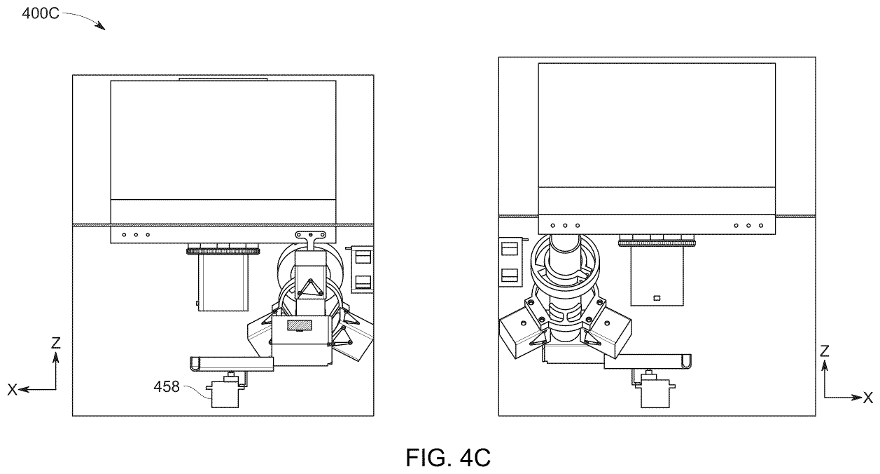

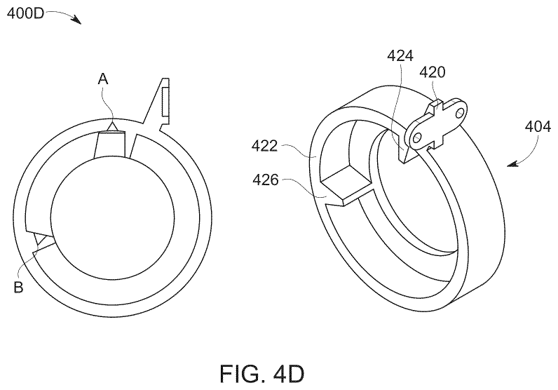

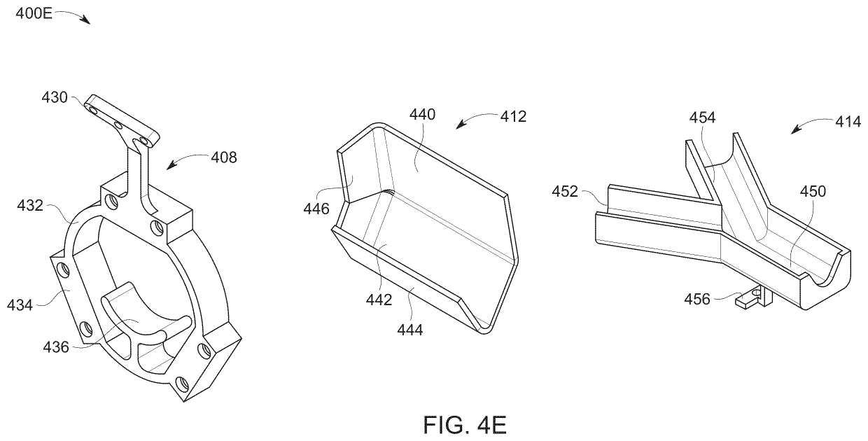

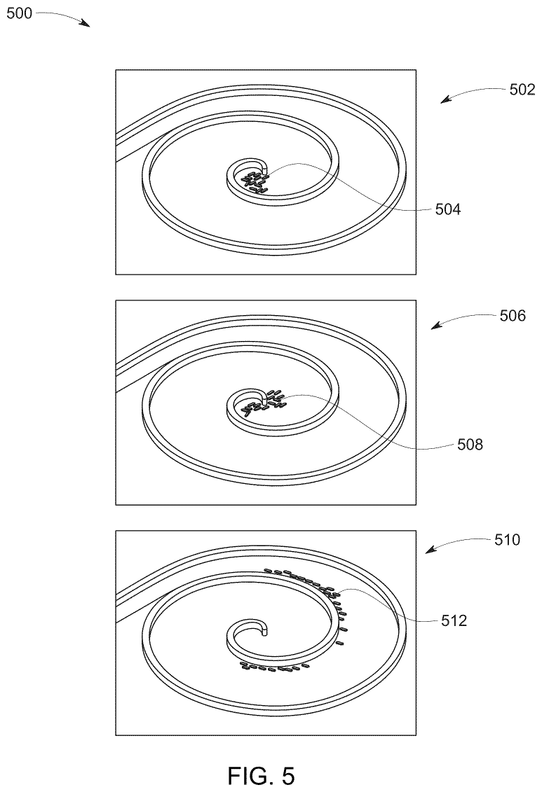

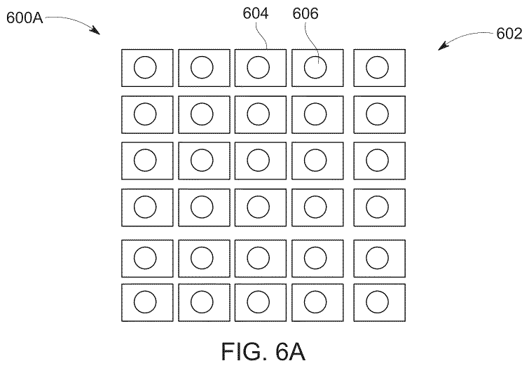

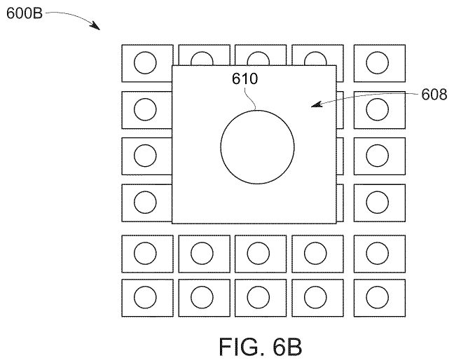

FIGS. 2A-2D, 3A-3D, 4A-4E, 5, and 6A-6b depict features of an example visual verification system 125. Features of the example visual verification system 125 may apply to features of FIG. 1 above. FIGS. 2A-2D depict external features of the example visual verification system 125 in diagram 200A, diagram 200B, diagram 200C, and diagram 200D. FIGS. 3A-3D depict a sorting features of the example visual verification system 125 in diagram 300A, diagram 300B, diagram 300C, and diagram 300D. FIGS. 4A-4E depict visual verification features of the example visual verification system 125 in diagram 400A, diagram 400B, diagram 400C, diagram 400D, and diagram 400E. FIG. 5 depicts an operation of the example visual verification system 125 in diagram 500. FIGS. 6A-6B depict a user interface of the example visual verification system 125 in diagram 600A and diagram 600B.

The visual verification system 125 may be operated by users of the environment 100, such as pharmacist or technicians of pharmacy system 120. The visual verification system 125 may be connected, e.g., via wired or wireless communication, to pharmacy system 120 and/or server 110 to provide relevant data therebetween. The visual verification system 125 may provide visual verification of medications, along with medication counting, medication stocking, and the like.

Turning to FIGS. 2A-2D, FIGS. 2A-2D depict external features of the example visual verification system 125. In particular, FIGS. 2A-2D may depict different orientation views of the visual verification system 125, such as a perspective view (FIG. 2A), top and bottom views (FIG. 2B), left and right views (FIG. 2C), and front and back views (FIG. 2D), with certain aspects labelled. In some cases, the visual verification system 125 may include a medication sorting device. The medication sorting device may include a medication entry 204, a first case 202, a moving part 208; a motor 418; a fixed part 206, and a second case 212.

The medication entry 204 may be configured to receive a plurality of medications. For instance, the medication entry 204 may be a funnel. The medication entry 204 may be replaceable (e.g., removeable), so that it can cleaned for different mediations. Generally, the medication entry 204 may have a first opening to receive the medications and a second opening to deposit the medications in a given location, as discussed herein. In some cases, the medication entry 204 may be a polygon (see FIG. 2A) or a cylinder (see funnel 204A in FIG. 3A). The medication entry 204 may be wider at a first end and narrower at a second end (e.g., near the moving part 208 and the fixed part 206).

The first case 202 may enclose a volume surrounding the moving part 208 and the fixed part 206. The first case 202 may be transparent. The first case 202 may reduce contamination while medications are sorted and/or processed. The first case 202 may be removable or fixed.

The moving part 208 may be configured to receive the plurality of medications from the medication entry 204 at a first location 210 and move the plurality of medications to a second location 220 (via opening 218). The moving part 208 may be a disk, as depicted in FIG. 3B. The moving part 208 may be replaceable (e.g., removeable), so that it can cleaned for different mediations. The opening 218 may be adjacent an edge of the moving part 208 and form a start of a channel (including a first catch 340, a tube 402 (having imaging zone 409), a second catch 412, and a sorting mechanism 414).

The motor 418 may be configured to move the moving part 208. The motor 418 may be a continuous or variable DC motor. The motor 418 may be controlled by a controller (e.g., processor, ASIC, and the like).

The fixed part 206 disposed adjacent the moving part and configured to interact with the plurality of medications, as the plurality of medications are moved to the second location, in order to generate a sequence of axially spaced apart medications. The fixed part 206 may be a spiral of certain dimensions, so as to interact with medications to axially arrange the medications in a spaced a part manner. The fixed part 206 may be replaceable (e.g., removeable), so that it can cleaned for different mediations. As discussed herein, the fixed part 206 may be various arrangements including fixed or variable radial extension from a center of the moving part 208 (including, e.g., a shoulder 308 of second fixed part 206A).

The second case 212 may be a housing for internal components 213. The second case 212 may include an opening 214, so that users may interact with the internal components 213. For instance, the users may move medication containers near one or more channels 452 or 454 (see, e.g., FIG. 4E) so as receive medications that have been processed by the visual verification system 125. The second case 212 may be opaque or transparent. The second case 212 may also provide structural rigidity to the internal components 213 and/or reduce contamination while medications are sorted and/or processed. The opening 214 may be closed by a door (not depicted), so as reduce contamination. The second case 212 may also have embedded therein or attached thereto a user interface 216. The user interface 216 may include various physical or digital controls of the visual verification system 125, such as a display or switches. In some cases, the user interface 216 may host the controller of the visual verification system 125. In other cases, the user interface 216 may provide user controls and/or present data to users from the controller.

In some cases, the second case 212 may include an opening 222 to circulate air via a fan 406. The fan 406 may be deposed adjacent the opening 222 to circulate air from an internal volume of the second case 212 to an external volume of the second case 212.

Turning to FIGS. 3A-3D, FIGS. 3A-3D depict a sorting features of the example visual verification system 125. In FIG. 3A, versions of the fixed part 206 may be depicted. The fixed part may include a first surface 302 and second surface 304 (see FIG. 3A) opposite the first surface 203 to form inner and outer surfaces of a spiral. The fixed part 206 may also include structural members 306 spanning (apart) from the surface of the moving part 208, so that medications may move below the structural members 306 while imparting structural rigidity to the walls of the fixed part 206. In some cases, the fixed part 206 may include a shoulder 308.

In FIG. 3B, the moving part 208 may be depicted. The moving part 208 may include a disk 310 (e.g., of a given diameter), engagement 312, and structural components 314. The engagement 312 may be configured to engage the motor 418 (e.g., a hex nut) to impart torque to the moving part 208. The structural components 314 may be reinforcing mass to support the disk 310.

In FIG. 3C, a tray 320 may be depicted. The tray 320 may retain motor 418 and the moving part 208 to the visual verification system 125 (e.g., directly or via main structure 330). The tray 320 may also include wall 324 to retain the fixed part 206. The tray 320 may also include an opening 326 corresponding to opening 218 of the fixed part 206 so as to pass medications through each of the openings into the channel. The tray 320 may also include an opening 322 to so that the motor 318 may be coupled to the moving part 208. The opening 322 may also retain the motor 318 and moving part 208 on a given axis. The tray 320 may also include an attachment 328 configured to (removable) engage the first catch 340.

In FIG. 3D, the main structure 330 may be depicted along with the first catch 340. The main structure 330 may provide structural attachments 334 for various components, including the motor 318 and the like, and include body 332. The first catch 340 may be a sloped surface 342 with retainer walls 344. The first catch 340 may be replaceable (e.g., removeable), so that it can cleaned for different mediations. The first catch 340 may also include attachment 346 that engages with attachment 328 of the tray 320.

Thus, as the motor 318 is operated (controlled) by the controller, medications may be organized into axially spaced apart medications from the first location to the second location and into the channel. After passing through the first catch 340 (as it is sloped downward), the axially spaced apart medications may pass into the tube 402.

Turning to FIGS. 4A-4E, the tube 402 may be partially or completely transparent. The tube 402 may be sloped at 5 degrees, 15 degrees, or up to 45 degrees from vertical. For instance, the tube 402 may be glass or partially glass (e.g., in the imaging zone 409). In some cases, the tube 402 may be removable from the visual verification system 125 (see, e.g., the retainer member 404 of FIG. 4D). The tube 402 may be generally cylindrical, but other shapes may be used, such as polygonal and the like. In some cases, the tube 402 may instead be a sloped surface with sufficiently high walls (e.g., not enclosed completely).

The imaging zone 409 may be in the field of view of one or more cameras 410. The one or more cameras 410 may held by a camera structure 408. The camera structure 408 may include an attachment 430 (that extends radially away from a ring 432) to the main structure 330, a support 436 for the tube 402 (that extends radially inward from the ring 432), and a plurality of camera mounts 434 (that extend radially away from the ring 432), each connected via the ring 432. In some cases, the plurality of camera mounts 434 may correspond to the number of one or more cameras (e.g., one, two, three, and the like). In some cases, there may be three cameras equally spaced around the tube 402 (e.g., spaced apart by 120 degrees). In some cases, there may be two cameras equally spaced around the tube 402 (e.g., spaced apart by 180 degrees); there may be four cameras equally spaced around the tube 402 (e.g., spaced apart by 90 degrees); generally, a number of cameras could be equally (or not-equally) spaced around the tube 402 (e.g., spaced apart by 360 degrees divided by number of cameras). In some cases, each of the one or more cameras could have different lens, or at least one of the cameras could have a different lens. In some cases, the lens may be swappable (e.g., from a first lens to a second lens). In this manner, different cameras (if lens are static) or different lens (on a same camera) could provide different resolutions and capturing radiuses.

The retainer member 404 be configured to lock and unlock so as retain the tube 402 in a fixed location or released. In some cases, a motion sensor (not depicted, e.g., infrared sensor) may be mounted on the retainer member 404. The motion sensor may be connected to the controller as discussed herein to indicate detection of medications in the tube 402. The retainer member 404 may include an attachment 420 to the main structure 330. The retainer member 404 may include a wall 422 (e.g., to stabilize the tube 402), and locking mechanisms 426 and 424. The mechanisms 426 and 424 may be protrusions radially inward to engage with corresponding fixtures on the tube 402.

The second catch 412 may be positioned at an end of the tube 402 (e.g., below and adjacent or abutting the tube 402). The second catch 412 may be replaceable (e.g., removeable), so that it can cleaned for different mediations. The second catch 412 may collect medications after imaging by the one or more cameras 410 and transport the medications to the sorting mechanism 414. The second catch 412 may include at least two walls to create a channel to gather and direct medications to the sorting mechanism 414. For instance, the second catch 412 may include at least two angled walls 442 and 444 to form the channel and a third covering wall 440 to block medications from bouncing over the second catch 412. The second catch 412 may also include an end wall 446 to block medications from traveling away from the sorting mechanism 414. The second catch 412 may be level (e.g., flat) or may be sloped from a first end (e.g., where medications are received) toward a second end (where medications are provided to the sorting mechanism 414. The end wall 448 may be provided at the first end. The second end may be open to the sorting mechanism 414.

The sorting mechanism 414 may include an entry channel 450 connected to a plurality of channels 452 and 454. The sorting mechanism 414 may be replaceable (e.g., removeable), so that it can cleaned for different mediations. While only two are depicted, two, three, four or more channels are envisioned so as to route medications to appropriate destinations based on identification of each medication and a count thereof. The sorting mechanism 414 may include a servo 458 (e.g., a motor, see FIGS. 4A and 4C) to control a channel gate 459 attached to attachment 456. The servo 458 may be controlled by the controller.

In some cases, the medication entry is a funnel configured to place the plurality of medications at the first location.

In some case, the moving part may be a disk, and the first location may be off center of the disk. For instance, the first location may be within 5%, 10%, 15% of the a radius of the disk.

In some cases, the fixed part may be a spiral that revolves around the disk. The spiral may revolve around the disk at least 360 degrees and radially extends away from the center of the disk at fixed or variable radius inches per degree. For instance, the rate of radial extension may be 2 inches per 360 degrees, 4 inches per 360 degrees, 6 inches per 360 degrees, and the like. In some cases, the spiral may start at the first location and end at the second location.

In some cases, the spiral may include a shoulder portion that breaks clumps and/or axially orientates medications in the axially spaced apart medications. For instance, the shoulder portion may extend radially inwardly at least 1 inch, 2 inches or more, and the like. In some cases, the shoulder portion may continuous (e.g., smoothly transitioned) or discontinuous (e.g., having change in radial distance at a same angle). In some cases, the shoulder portion is proximate the second location. For instance, the should portion may be within 30 degrees, 45 degrees, of up to 180 degrees from the second location. In these cases, the shoulder portion may extend radially in toward the center of the disk.

In some cases, the visual verification system (at the second location via the one or more cameras) may be configured to confirm a medication identity for each medication in the sequence of axially spaced apart medications.

In some cases, the visual verification system may include a sorting mechanism to direct each medication in the sequence of axially spaced apart medications into at least one channel of a plurality of channels. In some cases, the plurality of channels may include a rejection channel, one or more prescription channels, and/or a stock channel. For instance, multiple prescriptions could be filled sequentially or in parallel for a same medication using counting and routing. In the case a medication is not identified, the medication may be routed to the rejection channel. In the case a medication is identified by a prescription channel is full (e.g., a container has an allocated amount), the medication may be routed to a stock channel.

In some cases, the visual verification system may include: a channel configured to receive a plurality of medications; one or more cameras, the one or more cameras being configured to generate a plurality of images of the plurality of medications as the medications travel through the channel; at least one processor; and at least one memory storing instructions to control the system. The system may be configured to: receive, from the one or more cameras, the plurality of images of the plurality of medications; determine, based on the plurality of images, features of at least a first medication of the plurality of medications; and determine an identity of at least the first medication based on the features.

For instance, the imaging zone may be transparent so that the one or more cameras are able to image all sides of the medication in the imaging zone. In this manner, the medication may be identified by the images of the medications.

In some cases, the system may include a motion sensor configured to detect a presence of the medication in the imaging zone. In this manner, the system may only image the field of view when medications are in the field of view and image processing may be omitted (e.g., that does not include medications). In this case, the system may be configured to transmit a command, in response to detecting the presence of the medication in the imaging zone, to the one or more cameras to capture the plurality of images of the medication.

In some cases, the system may include a scale to weight different numbers of medication. The scale may be connected to the controller to verify inventory and/or amount of medication dispensed.

In some cases, the system may be configured to select at least one image of a portion of the medication. For instance, the camera(s) may provide 10s, 100s, or 1000s of images of the medications while in the imaging zone. The system may select an image of each discrete medication (even if multiple medications are in the imaging zone at one frame). For instance, the system may use selection criteria (e.g., blurriness, features extracted, occlusion, and the like) to select the at least one image.

In some cases, to determine the features of the medication, the system may be configured to process the at least one image of the portion of the medication through a neural network to thereby extract the features. In this case, the neural network may be trained on training data of training images and known features of the training images. The neural network may then, at inference, determine the features using the trained weights, biases, and the like. In some cases, the features may include text code on surface of the medication, color pattern of the surface of the medication, shape of the medication, scouring features of the medication, or other markings.

In some cases, to determine the identity of the medication, the system may be configured to determine whether the determined features match known features of a medication. For instance, the system may retrieve the known features of the medication based on a user input on a user interface, the user input indicating a medication. In some cases, the system may automatically determine a matching medication from a database of features. In some cases, the system may perform both matching processes to determine if the matches agree with each other.

In some cases, the system may be configured to store/output a verification record to be displayed to a user. The verification record may include prescription fill data (e.g., time, person, patient, intended medication, and the like) and at least one or combinations of: the plurality of images, subsets thereof, and/or a video of the medication (as it is being filled).

In some cases, the plurality of medications may be intended to fill a prescription, and the system may be configured to generate at least one image for each medication in the prescription. In this case, the verification record may include the at least one image for each medication in the prescription. In some cases, the plurality of medications may be intended for inventory control, and the system may be configured to generate at least one image of each medication in the inventory of a medication, and the system may automatically transmit relevant data (e.g., quantity, weight, time, and the like) to the pharmacy system 120.

In some cases, the system includes the sorting mechanism to direct each medication in a sequence of axially spaced apart medications into at least one channel of a plurality of output channels. For instance, the system may be configured to control the sorting mechanism based on the determined identity of the medication (e.g., based on the images).

In some cases, the system is configured to: detect the sequence of axially spaced apart medications (e.g., using the motion sensor) and determine a first time for each medication in the sequence of axially spaced apart medications based on the detection. For instance, the system may assign to each medication a timestamp when it was detected.

The system may then determine a second time frame for each medication in the sequence of axially spaced apart medications for when each medication will be in a sort zone of the sorting mechanism; and control the sorting mechanism based on the determined identity of each medication and the determined second time frame. For instance, the system may be configured to determine the second time frame based on a travel path from the channel to the sorting mechanism (e.g., using known distance travelled along channel, gravity, friction of medications on surfaces, and the like).

In some cases, the plurality of images of the plurality of medications includes at least one image that has at least two medications in a field of view of the one or more cameras. In this case, the system may be configured to: detect and distinguish between the at least two medications in order to determine the sequence of axially spaced apart medications. For instance, image segmentation and detection bounding using computer vision may detect each distinct medication and assign each a different order of the axially spaced apart medications.

In some cases, the system may include a bottle/package scanner. For instance, the bottle/package scanner may be a camera (that can read barcodes or QR codes), barcode scanner, or QR code scanner to detect and identify bottle/packages and automatically load relevant data to the system (e.g., NDC, drug name, strength, expiration dates, lot number). For instance, the system may automatically find relevant prescriptions to be filled using a same medication and automatically fulfill a prescribed amount, or suggest to fulfill that prescribed amount. The bottle/package scanner may be attached an exterior of the second case 212.

In some cases, the system may include external arm with a camera mount. The external arm with a camera mount may be configured to take pictures of packages and stock bottles, to create a record trail from medication entry to exit (e.g., time stamped record). In some cases, the external arm may be fixed (e.g., at a spaced a part distance from exterior of the second case 212). In some cases, the external arm may be configured to move (e.g., swing, pivot, telescope, and the like) so that the camera may be moved relative to the second case 212).

In some cases, the system may include fillers for prescription bottles with an infrared sensor to double count the medications as the medications are sorted into respective containers via respective channels.

In some cases, the system includes a bottom frame plate. The bottom frame plate may be configured to support the entire system and provide structural rigidity.

In some cases, the system may include a camera over the reject container (e.g., tray). The camera over the reject container may document medications that are damaged or not the same as other medications, thereby generating a record of inventory loss and/or providing a preventive audit trail to reduce risk.

FIG. 5 depicts an operation of the example visual verification system 125. At time 502, the medications may be a clump 504 at the first location. At time 506, the moving part may have moved the medications into spaced apart medications 508, thereby separating the clump 504 e.g. by interacting the medications with the moving part and the fixed part. At time 510, the medications may be ordered into a sequence of axially spaced apart medications 512, ready to be processed by the visual verification system (e.g., cameras and sorting mechanism).

FIGS. 6A-6B depict a user interface of the example visual verification system 125. FIG. 6A may depict a first user interface 602 depicting a plurality of images 604 depicted first portion 606 (e.g., a same side or portion) of each medication of a prescription. The plurality of images may be formatted into a grid corresponding to a number of medications (e.g., 15, 30, 45, as may be standard for a medication). Each image may be user selectable (e.g., by a user input) to display a different side or portion of the medication. FIG. 6B may depict the change in user interface in response to a user input selecting a particular medication to display an image 608 of the second side/different portion 610. In this manner, users of the system may visually confirm individual medications without interacting directly with the medication, thereby increasing safety and creating a record of the medication fulfillment.

Medication Prescription Verification

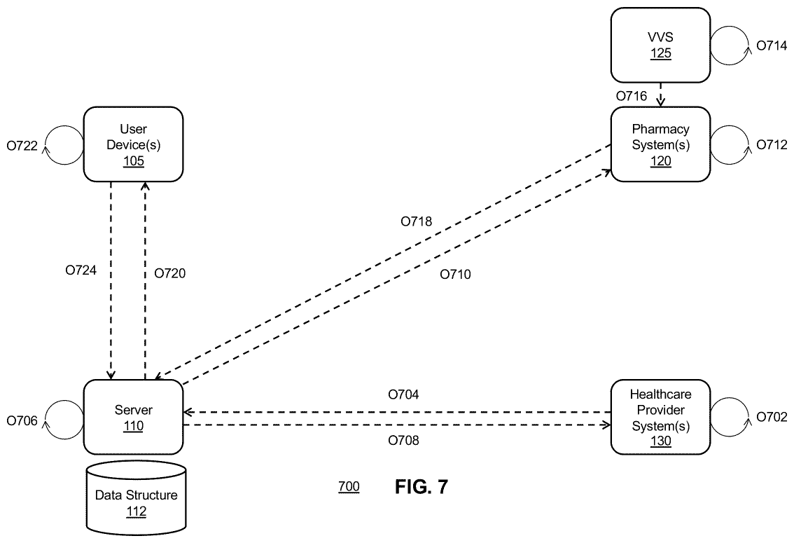

FIG. 7 depicts operations 0702 through 0724 for medication prescription verification through to medication fulfillment in block diagram 700. The operations 0702 through 0724 may occur on or between the user device(s) 105, the server 110, the pharmacy system(s) 120, the visual verification system 125, and the healthcare provider system(s) 130. The operations 0702 through 0724 may apply to features of FIGS. 1, 2A-2D, 3A-3D, 4A-4E, 5, and 6A-6b above.

In operation 0702, the healthcare provider system 130 may receive a user input or sequence of user inputs. The healthcare provider system 130 may process the user input(s) locally and update a user interface accordingly. Additionally, the healthcare provider system 130 may determine whether a user is entering prescription data into the system. For instance, the healthcare provider system 130 may determine whether the user is on prescription entry interface and entering, e.g., (1) patient data for a prescription, (2) doctor data for the prescription, and/or (3) medication data for the prescription. If so, the healthcare provider system 130 may determine the user is entering prescription data; if not, the healthcare provider system 130 may perform actions corresponding to the user input(s) (e.g., gather and present patient data, patient communications, healthcare records, or medical information, and the like). In response to determining the user is on prescription entry interface and entering prescription data, the healthcare provider system 130 may periodically (e.g., while a user is typing in a field) or in response to triggers (e.g., switching fields, a save action, a complete action, and the like) generate a medication prescription verification request. The medication prescription verification request may include prescription data (as currently entered at the time of transmission, in accordance with the transmission condition) for a proposed prescription for a patient. The medication prescription verification request may include some or all of a medication identifier, a usage indication, prescription directions, a quantity amount, a medication strength, patient data for the patient, and doctor data for the prescribing user from the prescription data (as the case may be, in the course of filling out prescription data).

In operation 0704, the healthcare provider system 130 may transmit the medication prescription verification request to the server 110. The server 110 may receive the medication prescription verification request.

In operation 0706, the server 110 may process the medication prescription verification request. For instance, the server 110 may extract one or combinations of a medication identifier, a usage indication, prescription directions, a quantity amount, a medication strength, patient data for the patient, and doctor data for the prescribing user from the prescription data. The server 110 may confirm various fields against existing records (e.g., in the data structure 112) and perform checks and/or recommendations, as discussed herein.

In some cases, the server 110 may confirm that a user matches the patient as indicated by the patient data, confirm that a doctor (or prescriber) matches the doctor data as indicated by the doctor data, that a medication identifier or name matches a known medication, and the like. If one or more of these are determined not to match a known patient, doctor, medication identifier or name, the server 110 may generate a result message indicating the issue (e.g., no patient by that name, or patient name does not ID, and the like) and/or with suggested solutions (e.g., correct spellings and the like). For instance, the server 110 may determine a user identifier that corresponds to the patient based on the patient data (e.g., by a extracting an ID from the patient data and matching it to records of the data structure 112), confirm that the name and, e.g., address of the patient in the data structure 112 match the name and address of the patient data in the request. Similarly, the data structure 112 may store names, addresses, or data regarding doctors (or prescribers) and verify that the doctor data in the request matches the records in the data structure 112. In the case that all data match (e.g., no errors), the server 110 may generate a result message indicating no issues (thereby closing the loop with the healthcare provider system 130. In some cases, the server 110 may not transmit the no error message (and the healthcare provider system 130 may presume, unless notified otherwise, that the data is correct or subject to future validation).

In some cases, the server 110 may perform a medication prescription verification. For instance, the server 110 may wait to perform the medication prescription verification once a sufficient amount of data has been entered into the system by the user. In this way, the server 110 may not try to process incomplete fields or fields that are not yet in a finished state. In these cases, the server 110 may determine a user identifier that corresponds to the patient based on the patient data by extracting an ID from the patient data and matching it to records of the data structure 112.

Based on the ID, the server 110 may retrieve active prescriptions and existing health conditions for the patient from the data structure 112 (e.g., based on user ID). Active prescriptions may be centrally managed by the server 110, so that the active prescriptions may be updated over time in response to data updates from one or more of the user device(s) 105, the server 110, the pharmacy system(s) 120, the visual verification system 125, and the healthcare provider system(s) 130. For instance, various different users of the user device(s) 105, the server 110, the pharmacy system(s) 120, the visual verification system 125, and the healthcare provider system(s) 130 may indicate a patient is taking a medication or is no longer taking a medication, and the server 110 may maintain a list of those actively-taking medications. The server 110 may also maintain, on a user basis, lists of previous medications (e.g., were taking but are no longer), lists of prescribed medications (e.g., those that have been prescribed but not acquired or started taking), lists of recalled medications (e.g., those that were prescribed but have since been recalled by a manufacturer of the medication), and the like. The existing health conditions may include diagnosed conditions (e.g., high-blood pressure, diabetes, and the like), test results (e.g., weight, height, blood pressure, and the like as measured by medical devices over time), activity records (e.g., as tracked and reported by activity trackers, such as smart watches and the like), and genetic tests/conditions (such as neurologic diseases or pharmacogenomic markers and the like).

The server 110 may perform an analysis to flag issues (if any) for verification by the medical provider. In this case, the server 110 may check both medical conflicts and availability conflicts, thereby increasing quality of care for patients, reducing time spent by users (e.g., re-entering or adjusting prescriptions after issues are discovered), and reducing delay to when a prescription can be fulfilled for a patient. The analysis may include one or combinations of checks to: (1) determine whether a medication corresponding to the medication identifier is under a recall flag or associated with the usage indication; (2) determine whether the medication has contraindications with the active prescriptions or interactions with the existing health conditions; (3) determine whether the prescription directions, the quantity amount, and the medication strength satisfy conditions associated with the medication; (4) determine whether the medication is a pharmacogenomically labelled medication; and/or (5) determine whether the medication, in the quantity amount and the medication strength, is available at a set of pharmacies.

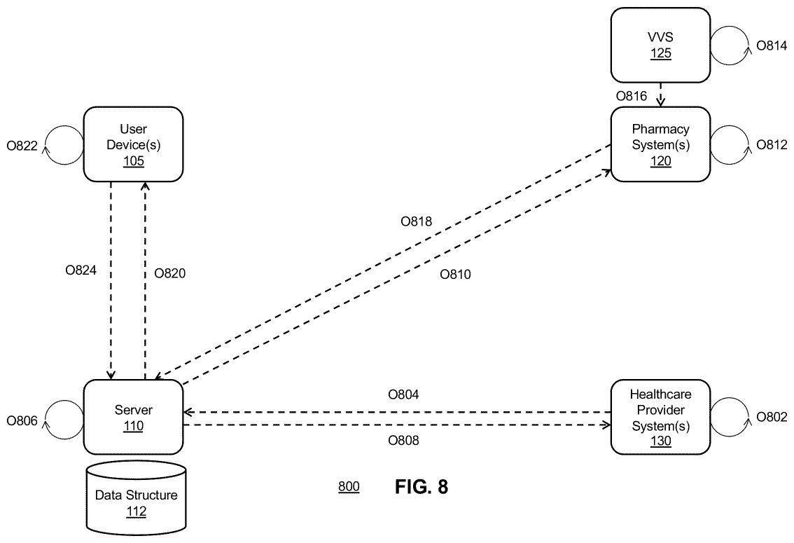

The server 110 may, in response to any check of the checks returning an issue, generate a result message. In this case, the result message may include indications of the issue (e.g., medication not available, medication has interaction, and the like) and/or recommendations. In some cases, the recommendations may be a different medication (as discussed with respect to FIG. 8) or it may be an indication that the medication is available at a certain location and the like.

The server 110 may, in response to no check of the checks returning an issue, generate a result message. In this case, the result message may include a verification that all entered data (as the system is currently aware) does not raise flags for verification. In some cases, the result message may indicate an option to submit the prescription. In some cases, the initial request included a request to submit the prescription if not issues were flagged.

In operation 0708, the server 110 may transmit the result message to the healthcare provider systems 130. As discussed above, the result message may indicate certain issues or no issues. In this manner, the server 110 and healthcare provider systems 130 may iteratively send data back and forth so that medication prescription fulfillment may be safe and efficient. Thus, the server 110 may transmit a notification to the healthcare provider systems 130 to receive a verification of the prescription data or a change in the prescription data. For instance, the healthcare provider may acknowledge the contraindications the issue presented by the server 110, but indicate to proceed, and the server 110 may document the acknowledgement and store a corresponding record. Similarly, the healthcare provider may adjust the prescription data to address the issue and the server 110 may re-run the check and provider further feedback. In this manner, prescription medications for patients is verified but also provided flexibility to provide quality of care.

In operation 0710, the server 110 may transmit a prescription request to the pharmacy system 120. The prescription request may include at least the patient data, doctor data, prescription data, and the user ID. The pharmacy system 120 may receive the prescription request.

In some cases, the server 110 may, in response to no checks returning an issue, transmit a prescription request to a prescription management system. The prescription management system may be a system to manage prescriptions between pharmacy systems 120, so that a prescription for the patient may be fulfilled at different pharmacy systems 120, thereby easing transfer of prescriptions and ease for the patients/caregivers. In some cases, the server 110 may be the prescription management system.

In operation 0712, the pharmacy system 120 may receive a user input or automatically confirm receipt of the prescription request (e.g., after checking records that the medication is in stock).

In operation 0714, a user of the visual verification system 125 may interact with the visual verification system 125. For instance, the visual verification system 125 may receive a list of to-be-filled prescriptions (e.g., from the server 110) and when a medication is scanned, automatically know to dispense certain amounts (or based on a user entered amount, and the like). The visual verification system 125 may then execute prescription fulfillment (e.g., filling a bottle), along with generating a record of images/videos of the prescription for visual verification by a user (e.g., pharmacist).

In operation 0716, the visual verification system 125 may transmit a prescription filled message to the pharmacy system 120. The pharmacy system 120 may use the prescription filled message to update inventory, re-order stock, and the like.

In operation 0718, the pharmacy system 120 may transmit a prescription filled message to the server 110. In some case, the pharmacy system 120 may relay the prescription filled message from the visual verification system 125 or be omitted. The prescription filled message may include record data (e.g., images/video, and the like), to be stored in the data structure 112. In some cases, the pharmacy system 120 may also store the record data (e.g., in a blockchain data storage architecture).

In operation 0720, the server 110 may transmit a prescription message to the user device associated with a patient. For instance, the patent (or caregiver) may be logged into an application or website and the user device may be authorized to receive notifications regarding prescriptions and/or healthcare data. In some cases, the prescription message is sent before the prescription request is sent to the pharmacy system 120, so the user may select a specific pharmacy system 120 to fulfill the prescription. In some cases, the prescription message is sent after the prescription request is sent to the pharmacy system 120. For instance, the specific pharmacy system 120 may be a default or pre-selected (e.g., by the user or doctor) pharmacy system 120 for the user and/or the medication.

In operation 0722, the user device 105 may receive user input(s) regarding the prescription. For instance, the user may indicate confirmation that the prescription is ready, request a change of pharmacy system 120, request a different medication or suggestion (e.g., due to cost), and the like. The user device 105 may then generate confirmation or request messages. The user device 105 may then generate and/or display data to the user in accordance with the user input(s) and/or data from the server 110.

In operation 0724, the user device 105 may transmit the confirmation or request messages to the server 110. The server 110 may receive the confirmation or request messages and respond accordingly (e.g., transfer prescriptions, provide data, and the like).

In some cases, to determine whether the medication corresponding to the medication identifier is under the recall flag or associated with the usage indication, the server 110 may be configured to: retrieve, periodically, recall data from a government entity. The recall data may be stored in the data structure 112. The recall data may indicate sets of lots of medications subject to a recall. The server 110 may update a recall data structure to indicate active recalled lots of medications; and determine whether the medication (of this prescription) is a part of a lot included in the active recalled lots of medications. For instance, the server 110 may match names, IDs, and/or lot numbers in the sets of lots of medications.

In some cases, to determine whether the medication has contraindications with the active prescriptions or interactions with the existing health conditions, the server 110 may be configured to: retrieve contraindications rules that indicate two or more medications should not be taken at a same time and retrieve interactions rules that indicate at least one medication should not be taken with specific existing health conditions. The contraindications rules and interactions rules may be stored in the data structure 112 and updated over time as domain knowledge and best practices change and evolve. The server 110 may apply the contraindications rules and the interactions rules to the medication, the active prescriptions, and the existing health conditions. For instance, matching medications, active prescriptions, and the existing health conditions, and the like may indicate a dangerous medication to prescribe so verification by the healthcare worker is desirable to ensure quality of care.

In some cases, to determine whether the prescription directions, the quantity amount, and the medication strength satisfy the conditions associated with the medication, the server 110 is configured to: retrieve quantity, direction, and strength rules for the medication. The quantity, direction, and strength rules for the medication may be stored in the data structure 112 and updated over time as domain knowledge and best practices change and evolve. The quantity, direction, and strength rules for the medication may, for instance, be ranges or values or classifications of actions that are deemed (by healthcare providers) as standard of care. The server 110 may apply the quantity, direction, and strength rules to the prescription directions, the quantity amount, and the medication strength to determine that the prescription directions, the quantity amount, and the medication strength are within the standard of care.

In some cases, to determine whether the medication is a pharmacogenomic medication, the system is configured to: retrieve, periodically, pharmacogenomic data from a government entity. The pharmacogenomic data may be stored in the data structure 112 and updated over time. The pharmacogenomic data may indicate drugs that have variable response for patients based on particular genotypes. The server 110 may update a pharmacogenomic data structure to indicate medications subject to pharmacogenomic adjustment; and determine whether the medication is a medication subject to pharmacogenomic adjustment. For instance, the server 110 may determine whether the user has already been tested for the genotype and, if so, whether to adjust the medication strength, etc.

In some cases, to determine whether the medication, in the quantity amount and the medication strength, is available at a set of pharmacies, the server 110 is configured to: retrieve, periodically or in real-time, inventory data for the set of pharmacies. The inventory data may be stored in, e.g., the data structure 112 or in the pharmacy system(s) 120. The inventory data may indicate type and amount of medication at each pharmacy system 120 (e.g., physical store). The server 110 may determine whether the medication is available at one or more pharmacies of the set of pharmacies based on the inventory data.

In some cases, the server 110 may be configured to: in response to receiving the verification of the prescription data (i.e., the issue is acknowledged but not changed per the healthcare provider), generate a record. The record may indicate the medical provider is aware of the flag for verification and document an explanation provided from the medical provider.

In some cases, the server 110 is configured to: in response to receiving the change in the prescription data, re-perform the analysis to flag for verification by the medical provider. In this manner, the healthcare provider may iteratively determine high quality of care prescriptions to patients without dealing with patient/pharmacy feedback loops.

Alternative Medication Recommendation

FIG. 8 depicts operations 0802 through 0824 for alternative medication recommendation through to medication fulfillment in block diagram 800. The operations 0802 through 0824 may occur on or between the user device(s) 105, the server 110, the pharmacy system(s) 120, the visual verification system 125, and the healthcare provider system(s) 130. The operations 0802 through 0824 may apply to features of FIG. 1, 2A-2D, 3A-3D, 4A-4E, 5, 6A-6b, and 7 above.

In operation 0802, the healthcare provider system 130 may receive a user input or sequence of user inputs. The healthcare provider system 130 may process the user input(s) locally and update a user interface accordingly. Additionally, the healthcare provider system 130 may determine whether a user is entering prescription data into the system. For instance, the healthcare provider system 130 may determine whether the user is on prescription entry interface and entering, e.g., (1) patient data for a prescription, (2) doctor data for the prescription, and/or (3) medication data for the prescription. If so, the healthcare provider system 130 may determine the user is entering prescription data; if not, the healthcare provider system 130 may perform actions corresponding to the user input(s) (e.g., gather and present patient data, patient communications, healthcare records, or medical information, and the like). In response to determining the user is on prescription entry interface and entering prescription data, the healthcare provider system 130 may periodically (e.g., while a user is typing in a field) or in response to triggers (e.g., switching fields, a save action, a complete action, and the like) generate a medication prescription verification request that includes a request for alternative medications. The medication prescription verification request may include prescription data (as currently entered at the time of transmission, in accordance with the transmission condition) for a proposed prescription for a patient. The medication prescription verification request may include some or all of a medication identifier, a usage indication, prescription directions, a quantity amount, a medication strength, patient data for the patient, and doctor data for the prescribing user from the prescription data (as the case may be, in the course of filling out prescription data).

In operation 0804, the healthcare provider system 130 may transmit the medication prescription verification request to the server 110. The server 110 may receive the medication prescription verification request.

In operation 0806, the server 110 may process the medication prescription verification request. For instance, the server 110 may extract one or combinations of a medication identifier, a usage indication, prescription directions, a quantity amount, a medication strength, patient data for the patient, and doctor data for the prescribing user from the prescription data. The server 110 may confirm various fields against existing records (e.g., in the data structure 112) and perform checks and/or recommendations, as discussed herein.

In some cases, the server 110 may confirm that a user matches the patient as indicated by the patient data, confirm that a doctor (or prescriber) matches the doctor data as indicated by the doctor data, that a medication identifier or name matches a known medication, and the like. If one or more of these are determined not to match a known patient, doctor, medication identifier or name, the server 110 may generate a result message indicating the issue (e.g., no patient by that name, or patient name does not ID, and the like) and/or with suggested solutions (e.g., correct spellings and the like). For instance, the server 110 may determine a user identifier that corresponds to the patient based on the patient data (e.g., by a extracting an ID from the patient data and matching it to records of the data structure 112), confirm that the name and, e.g., address of the patient in the data structure 112 match the name and address of the patient data in the request. Similarly, the data structure 112 may store names, addresses, or data regarding doctors (or prescribers) and verify that the doctor data in the request matches the records in the data structure 112. In the case that all data match (e.g., no errors), the server 110 may generate a result message indicating no issues (thereby closing the loop with the healthcare provider system 130. In some cases, the server 110 may not transmit the no error message (and the healthcare provider system 130 may presume, unless notified otherwise, that the data is correct or subject to future validation).

In some cases, the server 110 may perform a medication prescription verification. For instance, the server 110 may wait to perform the medication prescription verification once a sufficient amount of data has been entered into the system by the user. In this way, the server 110 may not try to process incomplete fields or fields that are not yet in a finished state. In these cases, the server 110 may determine a user identifier that corresponds to the patient based on the patient data by extracting an ID from the patient data and matching it to records of the data structure 112.

Based on the ID, the server 110 may retrieve active prescriptions and existing health conditions for the patient from the data structure 112 (e.g., based on user ID). Active prescriptions may be centrally managed by the server 110, so that the active prescriptions may be updated over time in response to data updates from one or more of the user device(s) 105, the server 110, the pharmacy system(s) 120, the visual verification system 125, and the healthcare provider system(s) 130. For instance, various different users of the user device(s) 105, the server 110, the pharmacy system(s) 120, the visual verification system 125, and the healthcare provider system(s) 130 may indicate a patient is taking a medication or is no longer taking a medication, and the server 110 may maintain a list of those actively-taking medications. The server 110 may also maintain, on a user basis, lists of previous medications (e.g., were taking but are no longer), lists of prescribed medications (e.g., those that have been prescribed but not acquired or started taking), lists of recalled medications (e.g., those that were prescribed but have since been recalled by a manufacturer of the medication), and the like. The existing health conditions may include diagnosed conditions (e.g., high-blood pressure, diabetes, and the like), test results (e.g., weight, height, blood pressure, and the like as measured by medical devices over time), activity records (e.g., as tracked and reported by activity trackers, such as smart watches and the like), and genetic tests/conditions (such as neurologic diseases or pharmacogenomic markers and the like).

The server 110 may perform an analysis to flag issues (if any) for verification by the medical provider. In this case, the server 110 may check both medical conflicts and availability conflicts, thereby increasing quality of care for patients, reducing time spent by users (e.g., re-entering or adjusting prescriptions after issues are discovered), and reducing delay to when a prescription can be fulfilled for a patient. The analysis may include one or combinations of checks to: (1) determine whether a medication corresponding to the medication identifier is under a recall flag or associated with the usage indication; (2) determine whether the medication has contraindications with the active prescriptions or interactions with the existing health conditions; (3) determine whether the prescription directions, the quantity amount, and the medication strength satisfy conditions associated with the medication; (4) determine whether the medication is a pharmacogenomically labelled medication; and/or (5) determine whether the medication, in the quantity amount and the medication strength, is available at a set of pharmacies.

The server 110 may, in response to any check of the checks returning an issue, generate a result message that includes an alternative medication. In this case, the result message may include indications of the issue (e.g., medication has interaction, and the like) and/or recommendations (e.g., alternative medication). In some cases, the recommendations may be a different medication or it may be an indication that the medication is available at a certain location and the like.

The server 110 may, in response to no check of the checks returning an issue, generate a result message. In this case, the result message may include a verification that all entered data (as the system is currently aware) does not raise flags for verification. In some cases, the result message may indicate an option to submit the prescription. In some cases, the initial request included a request to submit the prescription if not issues were flagged.

In operation 0808, the server 110 may transmit the result message to the healthcare provider systems 130. As discussed above, the result message may indicate certain issues or no issues. In this manner, the server 110 and healthcare provider systems 130 may iteratively send data back and forth so that medication prescription fulfillment may be safe and efficient. Thus, the server 110 may transmit a notification to the healthcare provider systems 130 to receive a verification of the prescription data or a change in the prescription data. For instance, the healthcare provider may acknowledge the contraindications the issue presented by the server 110, but indicate to proceed, and the server 110 may document the acknowledgement and store a corresponding record. Similarly, the healthcare provider may adjust the prescription data to address the issue and the server 110 may re-run the check and provider further feedback. In this manner, prescription medications for patients is verified but also provided flexibility to provide quality of care.

In operation 0810, the server 110 may transmit a prescription request to the pharmacy system 120. The prescription request may include at least the patient data, doctor data, prescription data, and the user ID. The pharmacy system 120 may receive the prescription request.

In some cases, the server 110 may, in response to no checks returning an issue, transmit a prescription request to a prescription management system. The prescription management system may be a system to manage prescriptions between pharmacy systems 120, so that a prescription for the patient may be fulfilled at different pharmacy systems 120, thereby easing transfer of prescriptions and ease for the patients/caregivers. In some cases, the server 110 may be the prescription management system.

In operation 0812, the pharmacy system 120 may receive a user input or automatically confirm receipt of the prescription request (e.g., after checking records that the medication is in stock).

In operation 0814, a user of the visual verification system 125 may interact with the visual verification system 125. For instance, the visual verification system 125 may receive a list of to-be-filled prescriptions (e.g., from the server 110) and when a medication is scanned, automatically know to dispense certain amounts (or based on a user entered amount, and the like). The visual verification system 125 may then execute prescription fulfillment (e.g., filling a bottle), along with generating a record of images/videos of the prescription for visual verification by a user (e.g., pharmacist).

In operation 0816, the visual verification system 125 may transmit a prescription filled message to the pharmacy system 120. The pharmacy system 120 may use the prescription filled message to update inventory, re-order stock, and the like.

In operation 0818, the pharmacy system 120 may transmit a prescription filled message to the server 110. In some case, the pharmacy system 120 may relay the prescription filled message from the visual verification system 125 or be omitted. The prescription filled message may include record data (e.g., images/video, and the like), to be stored in the data structure 112. In some cases, the pharmacy system 120 may also store the record data (e.g., in a blockchain data storage architecture).

In operation 0820, the server 110 may transmit a prescription message to the user device associated with a patient. For instance, the patent (or caregiver) may be logged into an application or website and the user device may be authorized to receive notifications regarding prescriptions and/or healthcare data. In some cases, the prescription message is sent before the prescription request is sent to the pharmacy system 120, so the user may select a specific pharmacy system 120 to fulfill the prescription. In some cases, the prescription message is sent after the prescription request is sent to the pharmacy system 120. For instance, the specific pharmacy system 120 may be a default or pre-selected (e.g., by the user or doctor) pharmacy system 120 for the user and/or the medication.

In operation 0822, the user device 105 may receive user input(s) regarding the prescription. For instance, the user may indicate confirmation that the prescription is ready, request a change of pharmacy system 120, request a different medication or suggestion (e.g., due to cost), and the like. The user device 105 may then generate confirmation or request messages. The user device 105 may then generate and/or display data to the user in accordance with the user input(s) and/or data from the server 110.

In operation 0824, the user device 105 may transmit the confirmation or request messages to the server 110. The server 110 may receive the confirmation or request messages and respond accordingly (e.g., transfer prescriptions, provide data, and the like).

In some cases, the recommendation for an alternative medication includes a different medication, a different prescription direction, a different quantity amount, a different medication strength, or a genotyping test. For instance, the server 110 may be configured to determine the different medication, the different prescription direction, the different quantity amount, and/or the different medication strength based on the medication, the usage indication, and alternative medication rules. The alternative medication rules may be stored in the data structure 112 and updated over time as domain knowledge and best practices change and evolve. For instance, the alternative medication rules may be based on usage indications, ICD10 codes, and/or domain knowledge. As an example, the alternative medication rules may indicate a first drug may have one or more second medications (e.g., pharmaceutical equivalents, branded, generic, or sub-composition, etc. of the first medication). The server 110 may use the ICD10 codes to determine substitutions, and usage indications to determine ranges, and domain knowledge to grow the corpus of knowledge over time (e.g., based on feedback from healthcare providers after suggesting different medications or ranges).

In some cases, in response to determining the medication is a part of a lot included in the active recalled lots, the server 110 may determine a different lot not in the active recalled lots of the medication provided by a different manufacturer is available. For instance, the server 110 may determine a lot (in inventory data) of the medication that is not listed on active recalled lots. The server 110 may then recommend the different lot of the medication provided by the different manufacturer as the different medication.

In some cases, in response to the contraindications rules and the interactions rules indicating a conflict between to the medication, the active prescriptions, and the existing health conditions, the server 110 may determine a non-conflicting medication based on the alternative medication rules; and recommend the non-conflicting medication as the different medication. For instance, the server 110 may determine an alternative based on the ICD10 codes that does not have the conflict between to the medication, the active prescriptions, and the existing health conditions.

In some cases, in response to the quantity, direction, and strength rules indicating a non-normal value or direction, the server 110 may determine a closest normal value or direction based on the quantity, direction, and strength rules; and recommend the closest normal value or direction as the different prescription direction, the different quantity amount, the different medication strength. For instance, the server 110 may determine a closest threshold (e.g., low or high threshold to suggest against a value that exceeds that low or high threshold), and the like. In the case of directions, the server 110 may suggest the most common (e.g., highest frequency) or all known methods to the healthcare provider.

In some cases, in response to the determining the medication is a medication subject to pharmacogenomic adjustment, the server 110 may determine the genotype testing based on the medication. For instance, the server 110 may match the medication to one that is subject to pharmacogenomic adjustment, and retrieve a genotype testing that can be used to determine an adjustment to the medication. Moreover, the server 110 may determine (e.g., from records in the data structure 112) whether the patient has already performed the genotype testing, and if so, the result of the genotype testing. The server 110 may then suggest relevant adjustments to the medication based on the result of the genotype testing (e.g., increase or decrease strength and the like). For instance, in response to obtaining a prescription verification request, the server 110 may determine the prescription data indicates a pharmacogenomically labeled prescription, and prompt or inform the healthcare provider to order: (1) a genetic test corresponding to the pharmacogenomically labeled prescription or (2) retrieve a prior genetic test. The server 110 may then parse the prior genetic test (or results of the new genetic test) for Clinical Pharmacogenetics Implementation Consortium (CPIC) guidelines based on therapy to determine alternatives. For instance, the server 110 may transmit a request to the CPIC guidelines API for data.

In some cases, in response to the determining the medication is not available at one or more pharmacies, the server 110 may determine an available medication based on the alternative medication rules and the inventory data; and recommend the available medication as the different medication. For instance, based on ICD10 codes, the server 110 may determine a different medication that is available at locations within a certain distance from a default or pre-selected pharmacy system 120.

In some cases, the server 110 may be further configured to: hold the prescription in a secure manner and release the prescription to a pharmacy chosen by the patient. For instance, the user may request the prescription be transferred to a different pharmacy system 120 (e.g., not within a same network of pharmacy enterprises) and the server 110 may securely receive the prescription from the first pharmacy and transfer it to the second pharmacy (based on instructions from the patient). In this manner, the server 110 may maintain a record trail from prescription being written, to fulfillment to the patient, to when the patient (or other user) indicates the user is no longer taking the prescription.

In some cases, the server 110 may be configured to: receive, from the user device 105, a request to change a fulfilling pharmacy to a new pharmacy. In this case, the server 110 may request and receive, from a pharmacy system 120 of the fulfilling pharmacy, a release to hold the prescription in a secure manner. The server 110 may then hold the prescription in the secure manner until the user indicates a new pharmacy. The server 110 may then (in response to the user indicating a new pharmacy) request and receive, from a pharmacy system 120 of the new pharmacy, an acknowledgement to fulfill the prescription. After receiving the acknowledgement, the server 110 may transmit a notification to the user device 105 that the prescription has been moved.

In some cases, the server 110 may be configured to: track the active prescriptions in a first list; track inactive prescriptions in a second list; track prescribed, but not yet filed prescriptions in a third list; track prescribed and filled, but not yet retrieved by the patient, prescriptions in a fourth list. The server 110 may be configured to move prescriptions between the first list, the second list, the third list, and the fourth list based on user inputs on user devices indicating prescriptions are prescribed, filled, retrieved, and ended. In some cases, the server 110 may be configured to provide the active prescriptions to medical providers in response to a request from the patient.

In some cases, the various rules described herein may be defined or based on medical guidelines. For instance, the medical guidelines may be issued by various medical organizations, such as, but not limited to American College of Cardiology (ACC) and the American Heart Association (AHA).

Computer System

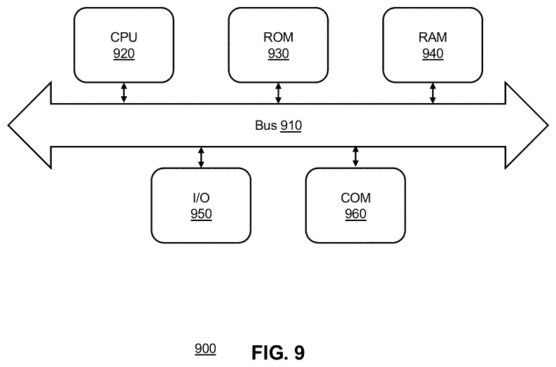

FIG. 9 depicts an example system that may execute techniques presented herein. FIG. 9 is a simplified functional block diagram of a computer that may be configured to execute techniques described herein, according to exemplary cases of the present disclosure. Specifically, the computer (or “platform” as it may not be a single physical computer infrastructure) may include a data communication interface 960 for packet data communication. The platform may also include a central processing unit (“CPU”) 920, in the form of one or more processors, for executing program instructions. The platform may include an internal communication bus 910, and the platform may also include a program storage and/or a data storage for various data files to be processed and/or communicated by the platform such as ROM 930 and RAM 940, although the system 900 may receive programming and data via network communications. The system 900 also may include input and output ports 950 to connect with input and output devices such as keyboards, mice, touchscreens, monitors, displays, etc. Of course, the various system functions may be implemented in a distributed fashion on a number of similar platforms, to distribute the processing load. Alternatively, the systems may be implemented by appropriate programming of one computer hardware platform.

The general discussion of this disclosure provides a brief, general description of a suitable computing environment in which the present disclosure may be implemented. In some cases, any of the disclosed systems, methods, and/or graphical user interfaces may be executed by or implemented by a computing system consistent with or similar to that depicted and/or explained in this disclosure. Although not required, aspects of the present disclosure are described in the context of computer-executable instructions, such as routines executed by a data processing device, e.g., a server computer, wireless device, and/or personal computer. Those skilled in the relevant art will appreciate that aspects of the present disclosure can be practiced with other communications, data processing, or computer system configurations, including: Internet appliances, hand-held devices (including personal digital assistants (“PDAs”)), wearable computers, all manner of cellular or mobile phones (including Voice over IP (“VoIP”) phones), dumb terminals, media players, gaming devices, virtual reality devices, multi-processor systems, microprocessor-based or programmable consumer electronics, set-top boxes, network PCs, mini-computers, mainframe computers, and the like. Indeed, the terms “computer,” “server,” and the like, are generally used interchangeably herein, and refer to any of the above devices and systems, as well as any data processor.

Aspects of the present disclosure may be embodied in a special purpose computer and/or data processor that is specifically programmed, configured, and/or constructed to perform one or more of the computer-executable instructions explained in detail herein. While aspects of the present disclosure, such as certain functions, are described as being performed exclusively on a single device, the present disclosure may also be practiced in distributed environments where functions or modules are shared among disparate processing devices, which are linked through a communications network, such as a Local Area Network (“LAN”), Wide Area Network (“WAN”), and/or the Internet. Similarly, techniques presented herein as involving multiple devices may be implemented in a single device. In a distributed computing environment, program modules may be located in both local and/or remote memory storage devices.

Aspects of the present disclosure may be stored and/or distributed on non-transitory computer-readable media, including magnetically or optically readable computer discs, hard-wired or preprogrammed chips (e.g., EEPROM semiconductor chips), nanotechnology memory, biological memory, or other data storage media. Alternatively, computer implemented instructions, data structures, screen displays, and other data under aspects of the present disclosure may be distributed over the Internet and/or over other networks (including wireless networks), on a propagated signal on a propagation medium (e.g., an electromagnetic wave(s), a sound wave, etc.) over a period of time, and/or they may be provided on any analog or digital network (packet switched, circuit switched, or other scheme).

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

### Terminology

The terminology used above may be interpreted in its broadest reasonable manner, even though it is being used in conjunction with a detailed description of certain specific examples of the present disclosure. Indeed, certain terms may even be emphasized above; however, any terminology intended to be interpreted in any restricted manner will be overtly and specifically defined as such in this Detailed Description section. Both the foregoing general description and the detailed description are exemplary and explanatory only and are not restrictive of the features, as claimed.

As used herein, the terms “comprises,” “comprising,” “having,” including,” or other variations thereof, are intended to cover a non-exclusive inclusion such that a process, method, article, or apparatus that comprises a list of elements does not include only those elements, but may include other elements not expressly listed or inherent to such a process, method, article, or apparatus.

In this disclosure, relative terms, such as, for example, “about,” “substantially,” “generally,” and “approximately” are used to indicate a possible variation of ±10% in a stated value.

The term “exemplary” is used in the sense of “example” rather than “ideal.” As used herein, the singular forms “a,” “an,” and “the” include plural reference unless the context dictates otherwise.

### Examples

Exemplary embodiments of the systems and methods disclosed herein are described in the numbered paragraphs below.

A1. A medication sorting device, comprising:
a medication entry configured to receive a plurality of medications;
a moving part configured to receive the plurality of medications from the medication entry at a first location and move the plurality of medications to a second location;
a motor configured to move the moving part; and
a fixed part disposed adjacent the moving part and configured to interact with the plurality of medications, as the plurality of medications are moved to the second location, in order to generate a sequence of axially spaced apart medications.

A2. The medication sorting device of A1, wherein the medication entry is a funnel configured to place the plurality of medications at the first location.

A3. The medication sorting device of any of A1-A2, wherein the moving part is a disk, and the first location is off center of the disk.

A4. The medication sorting device of any of A1-A3, wherein the fixed part is a spiral that revolves around the disk.

A5. The medication sorting device of A4, wherein the spiral revolves around the disk at least 360 degrees and radially extends away from a center of the disk at fixed or variable radius inches per degree.

A6. The medication sorting device of A4, wherein the spiral starts at the first location and ends at the second location.

A7. The medication sorting device of A4, wherein the spiral includes a shoulder portion that breaks clumps and/or axially orientates medications in the axially spaced apart medications.

A8. The medication sorting device of A7, wherein the shoulder portion is proximate the second location.

A9. The medication sorting device of A7, wherein the shoulder portion extends radially in toward the center of the disk.

A10. The medication sorting device of any of A1-A9, further includes a visual verification system at the second location to confirm a medication identity for each medication in the sequence of axially spaced apart medications.

A11. The medication sorting device of any of A1-A10, further includes a sorting mechanism to direct each medication in the sequence of axially spaced apart medications into at least one channel of a plurality of channels.

A12. The medication sorting device of A1, wherein the plurality of channels includes a rejection channel, a prescription channel, and a stock channel.

B1. A system for medication visual verification, comprising:
a channel configured to receive a plurality of medications;
one or more cameras, the one or more cameras being configured to generate a plurality of images of the plurality of medications as the medications travel through the channel;
at least one processor; and
at least one memory storing instructions, wherein the system is configured to:
   receive, from the one or more cameras, the plurality of images of the plurality of medications;
   determine, based on the plurality of images, features of at least a first medication of the plurality of medications; and
   determine an identity of at least the first medication based on the features.

B2. The system of B1, further includes an imaging zone, and the imaging zone is transparent so that the one or more cameras are able to image all sides of the medication in the imaging zone.

B3. The system of any of any of B1-B2, further includes a motion sensor configured to detect a presence of the medication in the imaging zone.

B4. The system of B3, wherein the system is configured to transmit a command, in response to detecting the presence of the medication in the imaging zone, to the one or more cameras to capture the plurality of images of the medication.

B5. The system of any of B1-B4, wherein the system is configured to select at least one image of a portion of the medication.

B6. The system of any of B1-B5, wherein, to determine the features of the medication, the system is configured to process the at least one image of the portion of the medication through a neural network to thereby extract the features.

B7. The system of B6, wherein the neural network is trained on training data of training images and known features of the training images.

B8. The system of any of B1-B7, wherein the features include text code on surface of the medication, color pattern of the surface of the medication, shape of the medication, scouring features of the medication, or other markings.

B9. The system of any of B1-B8, wherein, to determine the identity of the medication, the system is configured to determine whether the determined features match known features of a medication.

B10. The system of B8, wherein the system retrieves the known features of the medication based on a user input on a user interface, the user input indicating a medication.

B11. The system of any of B1-B10, wherein the system is configured to output a verification record to be displayed to a user, the verification record including prescription fill data and at least one or combinations of: the plurality of images, subsets thereof, and/or a video of the medication.

B12. The system of B11, wherein the plurality of medications is intended to fill a prescription, and the system is configured to generate at least one image for each medication in the prescription, and the verification record comprises the at least one image for each medication in the prescription.

B13. The system of any of B1-B12, wherein the system includes a sorting mechanism to direct each medication in a sequence of axially spaced apart medications into at least one output channel of a plurality of output channels.

B14. The system of B13, wherein the system is configured to control the sorting mechanism based on the determined identity of the medication.

B15. The system of B14, wherein the system is configured to: detect the sequence of axially spaced apart medications; determine a first time for each medication in the sequence of axially spaced apart medications based on the detection; determine a second time frame for each medication in the sequence of axially spaced apart medications for when each medication will be in a sort zone of the sorting mechanism; and control the sorting mechanism based on the determined identity of each medication and the determined second time frame.

B16. The system of B15, wherein the system is configured to determine the second time frame based on a travel path from the channel to the sorting mechanism.

B17. The system of B15, wherein the plurality of images of the plurality of medications includes at least one image that has at least two medications in a field of view of the one or more cameras, and the system is configured to: detect and distinguish between the at least two medications in order to determine the sequence of axially spaced apart medications.

C1. A system for prescription data verification, comprising:
at least one processor; and
at least one memory storing instructions that, when executed by the at least one processor, cause the system to:
   receive, from a user device associated with a medical provider, prescription data for a proposed prescription for a patient;
   extract a medication identifier, usage indication, prescription directions, a quantity amount, a medication strength, and patient data for the patient from the prescription data;
   determine a user identifier that corresponds to the patient based on the patient data;
   retrieve active prescriptions and existing health conditions for the patient;
   perform an analysis to flag for verification by the medical provider, including one or combinations of checks to:
      determine whether a medication corresponding to the medication identifier is under a recall flag or associated with the usage indication;
      determine whether the medication has contraindications with the active prescriptions or interactions with the existing health conditions;
      determine whether the prescription directions, the quantity amount, and the medication strength satisfy conditions associated with the medication;
      determine whether the medication is a pharmacogenomically labelled medication; and/or
      determine whether the medication, in the quantity amount and the medication strength, is available at a set of pharmacies;
   in response to any check of the checks returning an issue, transmit a notification to the user device associated with the medical provider to receive a verification of the prescription data or a change in the prescription data; and
   in response to no checks returning an issue, transmit a prescription request to a prescription management system, so that a prescription for the patient may be fulfilled.

C2. The system of C1, wherein, to determine whether the medication corresponding to the medication identifier is under the recall flag or associated with the usage indication, the system is configured to: retrieve, periodically, recall data from a government entity, the recall data indicating sets of lots of medications subject to a recall; update a recall data structure to indicate active recalled lots of medications; and determine whether the medication is a part of a lot included in the active recalled lots of medications.

C3. The system of any of C1-C2, wherein, to determine whether the medication has contraindications with the active prescriptions or interactions with the existing health conditions, the system is configured to: retrieve contraindications rules that indicate two or more medications should not be taken at a same time; retrieve interactions rules that indicate at least one medication should not be taken with specific existing health conditions; and apply the contraindications rules and the interactions rules to the medication, the active prescriptions, and the existing health conditions.

C4. The system of any of C1-C3, wherein, to determine whether the prescription directions, the quantity amount, and the medication strength satisfy the conditions associated with the medication, the system is configured to: retrieve quantity, direction, and strength rules for the medication; and apply the quantity, direction, and strength rules to the prescription directions, the quantity amount, and the medication strength.

C5. The system of any of C1-C4, wherein, to determine whether the medication is a pharmacogenomic medication, the system is configured to: retrieve, periodically, pharmacogenomic data from a government entity, the pharmacogenomic data indicating drugs that have variable response for patients based on particular genotypes; update a pharmacogenomic data structure to indicate medications subject to pharmacogenomic adjustment; and determine whether the medication is a medication subject to pharmacogenomic adjustment.

C6. The system of any of C1-C5, wherein, to determine whether the medication, in the quantity amount and the medication strength, is available at a set of pharmacies, the system is configured to: retrieve, periodically or in real-time, inventory data for the set of pharmacies; and determine whether the medication is available at one or more pharmacies of the set of pharmacies based on the inventory data.

C7. The system of any of C1-C6, wherein the system is configured to: in response to receiving the verification of the prescription data, generate a record indicating the medical provider is aware of the flag for verification and document an explanation provided from the medical provider.

C8. The system of any of C1-C7, wherein the system is configured to: in response to receiving the change in the prescription data, re-perform the analysis to flag for verification by the medical provider.

D1. A system for recommending alternative medications, comprising:
at least one processor; and
at least one memory storing instructions that, when executed by the at least one processor, cause the system to:
   receive, from a user device associated with a medical provider, prescription data for a proposed prescription for a patient;
   extract a medication identifier, usage indication, prescription directions, a quantity amount, a medication strength, and patient data for the patient from the prescription data;
   determine a user identifier that corresponds to the patient based on the patient data;
   retrieve active prescriptions and existing health conditions for the patient;
   perform an analysis to flag for verification by the medical provider, including one or combinations of checks to:
      determine whether a medication corresponding to the medication identifier is under a recall flag or associated with the usage indication;
      determine whether the medication has contraindications with the active prescriptions or interactions with the existing health conditions;
      determine whether the prescription directions, the quantity amount, and the medication strength satisfy conditions associated with the medication;
      determine whether the medication is a pharmacogenomically labelled medication; and/or
      determine whether the medication, in the quantity amount and the medication strength, is available at a set of pharmacies;
   in response to any check of the checks returning an issue, transmit a notification to the user device associated with the medical provider to recommend a different medication, a different prescription direction, a different quantity amount, a different medication strength, or a genotyping test; and
   in response to no checks returning an issue, transmit a prescription request to a prescription management system, so that a prescription for the patient may be fulfilled.

D2. The system of D1, wherein the system is configured to determine the different medication, the different prescription direction, the different quantity amount, and/or the different medication strength based on the medication, the usage indication, and alternative medication rules.

D3. The system of D2, wherein the alternative medication rules are based on usage indications, ICD10 codes, or domain knowledge.

D4. The system of any of D1-D3, wherein, to determine whether the medication corresponding to the medication identifier is under the recall flag or associated with the usage indication, the system is configured to: retrieve, periodically, recall data from a government entity, the recall data indicating sets of lots of medications subject to a recall; update a recall data structure to indicate active recalled lots of medications; determine whether the medication is a part of a lot included in the active recalled lots of medications; in response to determining the medication is a part of a lot included in the active recalled lots, determine a different lot not in the active recalled lots of the medication provided by a different manufacturer is available; recommend the different lot of the medication provided by the different manufacturer as the different medication.

D5. The system of any of D1-D4, wherein, to determine whether the medication has contraindications with the active prescriptions or interactions with the existing health conditions, the system is configured to: retrieve contraindications rules that indicate two or more medications should not be taken at a same time; retrieve interactions rules that indicate at least one medication should not be taken with specific existing health conditions; apply the contraindications rules and the interactions rules to the medication, the active prescriptions, and the existing health conditions; and in response to the contraindications rules and the interactions rules indicating a conflict between to the medication, the active prescriptions, and the existing health conditions, determine a non-conflicting medication based on the alternative medication rules; and recommend the non-conflicting medication as the different medication.

D6. The system of any of D1-D5, wherein, to determine whether the prescription directions, the quantity amount, and the medication strength satisfy the conditions associated with the medication, the system is configured to: retrieve quantity, direction, and strength rules for the medication; apply the quantity, direction, and strength rules to the prescription directions, the quantity amount, and the medication strength; in response to the quantity, direction, and strength rules indicating a non-normal value or direction, determine a closest normal value or direction based on the quantity, direction, and strength rules; and recommend the closest normal value or direction as the different prescription direction, the different quantity amount, the different medication strength.

D7. The system of any of D1-D6, wherein, to determine whether the medication is a pharmacogenomically labelled medication, the system is configured to: retrieve, periodically, pharmacogenomic data from a government entity, the pharmacogenomic data indicating drugs that have variable response for patients based on particular genotypes; update a pharmacogenomic data structure to indicate medications subject to pharmacogenomic adjustment; determine whether the medication is a medication subject to pharmacogenomic adjustment; and in response to the determining the medication is a medication subject to pharmacogenomic adjustment, determine the genotype testing based on the medication.

D8. The system of any of D1-D7, wherein, to determine whether the medication, in the quantity amount and the medication strength, is available at the set of pharmacies, the system is configured to: retrieve, periodically or in real-time, inventory data for the set of pharmacies; determine whether the medication is available at one or more pharmacies of the set of pharmacies based on the inventory data; and in response to the determining the medication is not available at one or more pharmacies, determine an available medication based on the alternative medication rules and the inventory data; and recommend the available medication as the different medication.

D9. The system of any of D1-D8, wherein the prescription management system is further configured to: hold the prescription in a secure manner and release the prescription to a pharmacy chosen by the patient.

D10. The system of any of D1-D9, wherein the prescription management system is configured to: receive, from a user device associated with the patient, a request to change a fulfilling pharmacy to a new pharmacy; request and receive, from a user device associated with the fulfilling pharmacy, a release to hold the prescription in a secure manner; hold the prescription in the secure manner; request and receive, from a user device associated with the new pharmacy, an acknowledgement to fulfill the prescription; and transmit a notification to the user device associated with the patient that the prescription has been moved.

D11. The system of any of D1-D10, wherein the system is configured to: track the active prescriptions in a first list; track inactive prescriptions in a second list; track prescribed, but not yet filed prescriptions in a third list; track prescribed and filled, but not yet retrieved by the patient, prescriptions in a fourth list; and move prescriptions between the first list, the second list, the third list, and the fourth list based on user inputs on user devices indicating prescriptions are prescribed, filled, retrieved, and ended.

D12. The system of any of D1-D11, wherein the system is further configured to provide the active prescriptions to medical providers in response to a request from the patient.

Other aspects of the disclosure will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A medication sorting device, comprising:
a medication entry configured to receive a plurality of medications;
a moving part configured to receive the plurality of medications from the medication entry at a first location and move the plurality of medications to a second location;
a motor configured to move the moving part;
a fixed part disposed adjacent the moving part and configured to interact with the plurality of medications, as the plurality of medications are moved to the second location, in order to generate a sequence of axially spaced apart medications.

2. The medication sorting device of claim 1, wherein the medication entry is a funnel configured to place the plurality of medications at the first location.

3. The medication sorting device of claim 1, wherein the moving part is a disk, and the first location is off center of the disk.

4. The medication sorting device of claim 3, wherein the fixed part is a spiral that revolves around the disk.

5. The medication sorting device of claim 4, wherein the spiral revolves around the disk at least 360 degrees and radially extends away from a center of the disk at fixed or variable radius inches per degree.

6. The medication sorting device of claim 4, wherein the spiral starts at the first location and ends at the second location.

7. The medication sorting device of claim 4, wherein the spiral includes a shoulder portion that breaks clumps and/or axially orientates medications in the axially spaced apart medications.

8. The medication sorting device of claim 7, wherein the shoulder portion is proximate the second location.

9. The medication sorting device of claim 7, wherein the shoulder portion extends radially in toward the center of the disk.

10. The medication sorting device of claim 1, further includes a visual verification system at the second location to confirm a medication identity for each medication in the sequence of axially spaced apart medications.

11. The medication sorting device of claim 1, further includes a sorting mechanism to direct each medication in the sequence of axially spaced apart medications into at least one channel of a plurality of channels.

12. The medication sorting device of claim 11, wherein the plurality of channels includes a rejection channel, a prescription channel, and a stock channel.

13. A system for medication visual verification, comprising:
a channel configured to receive a plurality of medications;
one or more cameras, the one or more cameras being configured to generate a plurality of images of the plurality of medications as the medications travel through the channel;
at least one processor; and
at least one memory storing instructions, wherein the system is configured to:
receive, from the one or more cameras, the plurality of images of the plurality of medications;
determine, based on the plurality of images, features of at least a first medication of the plurality of medications; and
determine an identity of at least the first medication based on the features.

14. The system of claim 13, further includes an imaging zone, and the imaging zone is transparent so that the one or more cameras are able to image all sides of the medication in the imaging zone.

15. The system of claim 13, further includes a motion sensor configured to detect a presence of the medication in the imaging zone.

16. The system of claim 15, wherein the system is configured to transmit a command, in response to detecting the presence of the medication in the imaging zone, to the one or more cameras to capture the plurality of images of the medication.

17. The system of claim 13, wherein the system is configured to select at least one image of a portion of the medication.

18. The system of claim 13, wherein, to determine the features of the medication, the system is configured to process the at least one image of the portion of the medication through a neural network to thereby extract the features.

19. The system of claim 18, wherein the neural network is trained on training data of training images and known features of the training images.

20. The system of claim 13, wherein the features include text code on surface of the medication, color pattern of the surface of the medication, shape of the medication, scouring features of the medication, or other markings.
